(19) United States
(12) Patent Application Publication
(10) Pub. No.: US 2024/0000899 A1
(43) Pub. Date: Jan. 4, 2024

(54) THERAPEUTIC USEFUL AGAINST ANTIMICROBIAL RESISTANT AGENTS

(71) Applicant: Tygrus, LLC, Troy, MI (US)

(72) Inventors: Paul Bundschuh, Austin, TX (US); Lawrence Carlson, North Branch, MI (US); Shawn Dolan, Sterling Heights, MI (US)

(21) Appl. No.: 18/200,760

(22) Filed: May 23, 2023

Related U.S. Application Data
(60) Provisional application No. 63/345,004, filed on May 23, 2022.

(51) Int. Cl. A61K 38/40; A61K 31/185; A61K 9/00; A61P 31/04

(52) U.S. Cl. CPC A61K 38/40; A61P 31/04; A61K 9/0078; A61K 31/185

(57) ABSTRACT

A pharmaceutically acceptable therapeutic inhalation fluid that is composed of a fluid carrier and a pharmaceutically acceptable acid formulation present in the fluid carrier in an amount sufficient to provide a solution pH between 1.5 and 2.5, the pharmaceutically acceptable acid formulation having anti-bacterial, and/or anti-viral, and/or anti-fungal activity and at least one antimicrobial peptide.

### CROSS-REFERENCE TO RELATED APPLICATION(S)

This application claims priority to and the benefit of U.S. Provisional Patent Application Ser. No. 63/345,004, filed May 23, 2022, the entire disclosure of which is hereby incorporated by reference.

### TECHNICAL FIELD

This disclosure relates to compositions suitable for use as therapeutic compositions that function as an anti-bacterial, anti-viral, and/or anti-fungal respiratory therapeutic or prophylactic and methods of treatment employing the same.

### BACKGROUND

The U.S. Centers for Disease Control and Prevention (CDC) and the World Health Organization (WHO) categorize antimicrobial-resistant (AMR) pathogens as a looming threat to human health. While AMR genes occur naturally in the environment, the use of antibiotics has selected for the presence of AMR genes. The lack of rapid diagnostic methods to identify bacterial pathogens and AMR genes in clinical settings has resulted in the often-unnecessary use of broad-spectrum antibiotics.

In February 2017, to focus and guide research and development related to new antibiotics, the WHO published its list of pathogens for which new antimicrobial development is urgently needed. Within this broad list, ESKAPE (Enterococcus faecium, Staphylococcus aureus, Klebsiella pneumoniae, Acinetobacter baumannii, Pseudomonas aeruginosa, and Enterobacter species) pathogens were designated “priority status”. ESKAPE pathogens have developed resistance mechanisms against oxazolidinones, lipopeptides, macrolides, fluoroquinolones, tetracyclines, -lactams, -lactam, -lactamase inhibitor combinations, and antibiotics that are the last line of defense, including carbapenems, glycopeptides, and clinically unfavorable polymyxins (De Oliveira, D. et al. (2020). Antimicrobial Resistance of ESKAPE pathogens, Clinical Microbiology Reviews).

It has been estimated that in 2019 4.95 million deaths were associated with bacterial AMR including 1.27 million deaths attributable to AMR. The leading indication of AMR mortality was respiratory infections. (Murray, C. et al. (2022). Global burden of bacterial antimicrobial resistance in 2019: a systematic analysis, Lancet). Therefore, there is an urgent global need for innovative antimicrobial therapies for ESKAPE and other AMR pathogens, particularly for respiratory infections.

Thus, it would be desirable to provide a composition and method for administering the same that can address infections, particularly those caused by one or more of the anti-microbial pathogens, including but not limited to one or more of the ESKAPE pathogens. It would also be desirable to provide a composition and method of administering the same that can address infections, particularly those caused by one or more of the ESKAPE pathogens presenting in the respiratory system of a subject. It would also be desirable to provide a composition and method of administering the same that can address infections, particularly those caused by one or more of the ESKAPE pathogens presenting in the respiratory system of a subject, in whole or in part via inhalation therapy.

### SUMMARY

A pharmaceutically acceptable therapeutic inhalation fluid that is composed of a fluid carrier and a pharmaceutically acceptable acid formulation present in the fluid carrier in an amount sufficient to provide a solution pH between 1.5 and 2.5, the pharmaceutically acceptable acid formulation having anti-bacterial, and/or anti-viral, and/or anti-fungal activity. The pharmaceutically acceptable therapeutic inhalation fluid further including at least one antimicrobial peptide either in admixture or co-administered therewith.

A method for treating a respiratory infection caused by bacterial, viruses, fungi and/or allergens that includes the steps of administering at least one dose of a pharmaceutically acceptable inhalation fluid of a pharmaceutically acceptable therapeutic inhalation fluid into contact with at least one region of the respiratory system of a subject, the dose introduced by one or more of a nebulizer, metered-dose inhalation device, vaporizer, humidifier, nasal irrigation device and the like. The pharmaceutically acceptable therapeutic inhalation fluid can be composed of a fluid carrier and a pharmaceutically acceptable acid formulation present in the fluid carrier in an amount sufficient to provide a solution pH between 1.5 and 2.5 and having anti-bacterial, and/or anti-viral, and/or anti-fungal activity.

### DETAILED DESCRIPTION

Disclosed herein are implementations of and method for treating or preventing a respiratory infection caused in whole or in part by a chronic, subacute, or acute respiratory infections caused by one or more antimicrobial microbial resistant pathogens. Also disclosed is a pharmaceutically acceptable therapeutic inhalation fluid composition that includes a fluid carrier and a pharmaceutically acceptable acidic component in a pharmaceutically acceptable composition. Where desired or required, the composition can have a pH of 2.5 or less and be utilized as an anti-bacterial, anti-viral and/or anti-fungal therapeutic agent for treating or preventing a respiratory infection. The method of treatment includes administering the low pH therapeutic solution as by inhalation of material processed by nebulizer, inhaler, nasal spray, nasal wash, vaporizer, humidifier and the like.

In certain embodiments, the pharmaceutically acceptable therapeutic inhalation fluid composition can be on that consists of the fluid carrier and the pharmaceutically acceptable acidic component with the pharmaceutically acceptable acidic component being one or more organic acids, one or more inorganic acids or mixtures thereof. In certain embodiments, the pharmaceutically acceptable therapeutic inhalation fluid composition can include pharmaceutically acceptable inorganic acid(s). In certain embodiments, the pharmaceutically acceptable inorganic acid can be selected from the group consisting of hydrochloric acid, sulfuric acid and mixtures thereof.

In certain embodiments, the pharmaceutically acceptable therapeutic inhalation fluid composition can include one or more active pharmaceutical ingredients in addition to the fluid carrier and pharmaceutically acceptable acidic component. In certain embodiments, non-limiting examples of such additional active pharmaceutical ingredient(s) include various adrenergic β₂ receptor agonists, steroids, non-steroidal anti-inflammatory compounds, muscarinic antagonists and mixtures thereof. In certain embodiments, non-limiting examples of such additional active pharmaceutical ingredient(s) include antimicrobial peptides. The present disclosure contemplates that, in certain embodiments, one or more of the additional active pharmaceutical ingredient(s) can be present in admixture with the carrier fluid and the pharmaceutically acceptable acidic component. The present disclosure also contemplates that the one or more additional active pharmaceutical ingredient(s) can be formulated for coadministration with the fluid carrier and pharmaceutically acceptable acidic component.

Respiratory illnesses that can be treated or prevented by the method and/or composition(s) as disclosed herein can include respiratory tract infections caused be one or more of a variety of infectious pathogens which can affect humans or animals or both. Respiratory illness that can be treated or prevented by the method as disclosed herein can include one or more chronic respiratory conditions. Respiratory illnesses that can be treated or prevented can be a combination of one or more chronic respiratory conditions and one or more acute respiratory infections. In certain embodiments respiratory tract infections can be either acute infections or chronic infections and can be caused by one or more pathogens. It is also contemplated that respiratory illnesses can be a combination of the chronic respiratory illness(es) and acute respiratory tract infection(s).

Chronic respiratory conditions as defined by the United States Center for Disease Control are defined broadly as conditions that last one year or more and require ongoing medical attention or curtail activities of daily living or both. Non-limiting examples of chronic respiratory illnesses that can be addressed by the method and/or composition disclose herein include chronic obstructive pulmonary disease, cystic fibrosis, asthma, or respiratory allergies.

Respiratory tract infections as that term in used in this disclosure is broadly defined as any infectious disease of the upper or lower respiratory tract. Upper respiratory tract infections can include, but are not limited to, the common cold, laryngitis, pharyngitis/tonsillitis, rhinitis, rhinosinusitis, and the like. Lower respiratory tract infections include bronchitis, bronchiolitis, pneumonia, tracheitis and the like.

In certain embodiments, the acute, subacute, or chronic respiratory infection can be caused by an antimicrobial-resistant pathogen that includes, but is not limited to Gram-negative bacteria, Gram-positive bacteria, viruses, fungi, parasites, and allergens. In certain embodiments, the respiratory infection can be caused particularly, in whole or in part, by one or more of the antimicrobial-resistant ESKAPE (Enterococcus faecium, Staphylococcus aureus, Klebsiella pneumoniae, Acinetobacter baumannii, Pseudomonas aeruginosa, and Enterobacter species) pathogens.

The pharmaceutically acceptable therapeutic inhalation fluid employed is one that comprises a carrier fluid and a pharmaceutically acceptable acid composition present in the carrier fluid in an amount sufficient to provide a solution pH between 1.5 and 2.5, the pharmaceutically acceptable acid composition exhibiting antimicrobial activity against at least one microbial pathogen when introduced into the respiratory system of a subject.

Pathogens responsible for respiratory tract infections that can be treated by the method and/or composition as disclosed herein can include one or more viral pathogens, one or more bacterial pathogens, one or more fungal pathogens as well as mixed pathogen infections arising from two or more of the classes discussed. In certain embodiments disclosed herein, the viral pathogen can be at least one of a coronavirus, an influenza virus, a parainfluenza virus, a respiratory syncytial virus (RSV), a rhinovirus, an adenovirus as well as combinations of two or more of the foregoing. It is also contemplated that the various viral strains causing infection in a patient can be pure strains or can be mixtures of various strains, types, subtypes and/or variants.

Coronaviruses that can be treated by the method and/or composition as disclosed herein include, but are not limited to, alpha coronaviruses, beta coronavirus as well as other emergent types. Coronaviruses, as that term is employed in this disclosure, are understood to be a group of related RNA viruses that cause disease, particularly respiratory tract infections in various mammalian and avian species. Coronaviruses that can be treated by the method and/or composition as disclosed herein include members of the subfamily Orthocoronavirinae in the family Coronaviridea. In certain embodiments, the method and/or composition as disclosed herein can be employed to treat or prevent respiratory infections in which the diseases-causing pathogen is a human coronavirus that is member of the family Coronaviridea selected from the group consisting of SARS-CoV-1 (2003), HCoV NL63(2004), HCoV HKU1 (2004), MERS-CoV (2013) SARS-CoV-2 (2019) and mixtures thereof. In certain embodiments the coronavirus can be a beta coronavirus selected from the group consisting of SARS-CoV, SARS-CoV-2, MERS-CoV, and mixtures thereof. In certain embodiments the method and/or composition as disclosed herein can be employed to treat or prevent respiratory infections in which the diseases-causing pathogen is an enveloped, positive-sense, single stranded RNA virus other than those mentioned.

Non-limiting examples of influenza viruses that can cause respiratory tract infections and can be treated by the method and/or compositions as disclosed herein can be negative-sense RNA viruses such as Orthomyxoviridae such as those from the genera: alphainfluenza, betainfluenza, deltainfluenza, gammainfluenza, thogotovirus and quaranjavirus. In certain embodiments, the influenza virus can be an alphainfluenza that expresses as a serotype such as H1N1, H1N2, H2N2, H3N1, H3N2, H3N8, H5N1, H5N2, H5N3, H5N8, H5N9, H7N1, H7N2, H7N4, N7N7, H7N9, H9N2, H10N7. Other expressions are also contemplated.

Non-limiting examples of parainfluenza viruses can be single-stranded, enveloped RNA viruses of the Paramyoviridae family. Non-limiting examples of human parainfluenza viruses include those in the genus Respirovirus and those in the genus Rubulavirus.

Non-limiting examples of respiratory syncytial viruses (RSV) are various medium sized (˜150 nm) enveloped viruses from the family Pneumvidae such as those in the genus Orthopneumovirus.

Non-limiting examples of rhinovirus that can be treated by the method and/or composition as disclosed herein include those with single-stranded positive sense RNA genomes that are composed of a capsid containing the viral protein(s). Rhinoviruses can be from the family Picovirus and the genus Enterovirus.

Non-limiting examples of adenoviruses include non-enveloped viruses such as those with an icosahedral nucleocapsid containing nucleic acid such as double stranded DNA. Viruses can be from the family Adenoviridae and genera such as Atadenovirus, Mastadenvirus, Siadenovirus, and the like.

It is also contemplated that the method and/or composition as disclosed herein can be used to treat respiratory infections caused by bacterial pathogens. Non-limiting examples of such bacterial pathogens include Streptococcus pneumoniae, Pseudomonas aeruginosa, Klebsiella pneumoniae, Haemophilus influenzae, Staphylococcus aureus, Moraxella catarrhalis, Streptococcus pyogenes, Mycobacterium tuberculosis, Mycobacterium avium-intracellulare (MAI), Mycobacterium terrae, and mixtures thereof.

The method and/or composition as disclosed herein can be used to treat respiratory infections caused by fungal pathogens presenting as single-pathogen fungal infections, multi-pathogen fungal infections or general mycosis with respiratory involvement. Non-limiting examples of fungal pathogens implicated in respiratory illnesses and infections include certain species from the genus Aspergillus, with A. fumigatus, A. flavus, and A. clavatus being non-limiting examples. Other examples of respiratory infections caused by fungal pathogens that can be treated by the method and/or compositions disclosed herein are respiratory infections involving infectious species of Cryptococcus, Rhizopus, Mucor, Pneumocystis, Candida, and the like.

In certain embodiments, there is disclosed as composition and method for treating or preventing an acute, subacute, or chronic respiratory infection caused by antimicrobial-resistant (AMR) pathogens including but not limited to Gram-negative bacteria, Gram-positive bacteria, viruses, fungi, parasites, and allergens. Non-limiting examples of such antimicrobial-resistant pathogens include collectively or individually, pathogens such as Enterococcus species, Staphylococcus species, Klebsiella species, Acinetobacter species, Pseudomonas species, and Enterobacter species. In certain embodiments, the antimicrobial-resistant pathogen that can be effectively treated by the method and/or composition as disclosed herein includes one or more of Enterococcus faecium, Staphylococcus aureus, Klebsiella pneumoniae, Acinetobacter baumannii, Pseudomonas aeruginosa, Enterobacter species.

In certain embodiments, the method and/or composition as disclosed herein can have a pH less than 2.5; less than 2.4; less than 2.3; less than 2.2 less than 2.1; less than 2.0; less than 1.9; less than 1.8; less than 1.7; less than 1.6; less than 1.5; with lower ranges being determined by the respiratory condition and health of the patient. In certain embodiments, the composition can have a have a pH between 1.5 and 2.5; between 1.6 and 2.5; between 1.7 and 2.5; between 1.8 and 2.5; between 1.9 and 2.5; between 2.0 and 2.5; between 2.1 and 2.5; between 2.2 and 2.5; between 2.3 and 2.5; between 2.4 and 2.5; between 1.5 and 2.4; between 1.6 and 2.4; between 1.7 and 2.4; between 1.8 and 2.4; between 1.9 and 2.4; between 2.0 and 2.4; between 2.2 and 2.4; between 2.2 and 2.4; between 2.3 and 2.5; between 2.4 and 2.5; between 1.5 and 2.4; between 1.6 and 2.4; between 1.7 and 2.4; between 1.8 and 2.4; between 1.9 and 2.4; between 2.0 and 2.4; between 2.1 and 2.4; between 2.2 and 2.4; between 1.5 and 2.3; between 1.6 and 2.3; between 1.7 and 2.3; between 1.8 and 2.3; between 1.9 and 2.3; between 2.0 and 2.3; between 2.1 and 2.3; between 2.2 and 2.3 between 1.5 and 2.2; between 1.6 and 2.2; between 1.7 and 2.2; between 1.8 and 2.2; between 1.9 and 2.2; between 2.0 and 2.2; between 2.1 and 2.2; between 1.5 and 2.0; between 1.6 and 2.0; between 1.7 and 2.0; between 1.8 and 2.0; between 1.9 and 2.0, between 1.5 and 1.9; between 1.6 and 1.9; between 1.7 and 1.9; between 1.8 and 1.9; between 1.5 and 1.8; between 1.6 and 1.8; between 1.7 and 1.8; between 1.5 and 1.7; between 1.6 and 1.7 between 1.5 and 1.6.

In the method as disclosed herein, the pharmaceutically acceptable fluid having a pH below 2.5 can be administered into contact with at least one region of the respiratory tract of the patient in need thereof can be administered by any therapeutically acceptable manner. In certain embodiments, the pharmaceutically acceptable fluid will be administered in a manner that permits or promotes uptake of at least a portion of the composition by patient inhalation. The pharmaceutically acceptable fluid can be introduced under pressure in certain embodiments.

The pharmaceutically acceptable fluid as disclosed herein can be introduced into contact with at least one region in the respiratory tract of the patient in the form of a gas, a fluid or a mixture of the two. In certain embodiments, the pharmaceutically acceptable fluid can also include one or more powders or micronized solids. The pharmaceutically acceptable fluid can be introduced into contact with at least a portion of the respiratory tract of the patient in the form a vapor, aerosol, spray, micronized mist, gas or the like. It is also contemplated that the pharmaceutically acceptable fluid can be administered as a gas, as dispersed nanoparticles in a gas, as micronized particles in a gas, as nanoparticles dispersed in a gas or the like.

The size particulate or droplet material composed of the pharmaceutically acceptable fluid that is introduced into contact with at least one region of the respiratory tract of the patient can be adjusted or tuned to increase contact with the desired region of the respiratory tract. The respective regions of the respiratory tract which the pharmaceutically acceptable fluid can contact can include nose, sinuses, throat, pharynx, larynx, epiglottis, trachea, bronchi, alveoli, or combinations of any of the foregoing. The size distribution of the particles/droplets can be tuned to address the location of greatest pathogen population. In certain embodiments, the at least one dose of a pharmaceutically acceptable fluid can be delivered into contact with the lower respiratory tract such as the bronchi, alveoli and the like in order to address infections localized in that region. In certain embodiments, the at least one dose of the pharmaceutically acceptable fluid can be delivered into contact with the upper respiratory tract such as the nose or nostrils, nasal cavity, mouth, pharynx, larynx and the like to address infections localized in this region.

As used herein the term “at least one dose of the pharmaceutically acceptable fluid” is defined as the delivery of between 0.5 ml and 30 ml of a pharmaceutically acceptable therapeutic inhalation fluid as disclosed herein comprising a fluid carrier and a pharmaceutically acceptable acid composition present in the carrier fluid in an amount sufficient to provide a solution pH between 1.5 and 2.5 in combination with an effective amount of at least one antimicrobial peptide over in an atomized, nebulized and/or vaporized state over and interval of 15 seconds to 30 minutes. In certain embodiments, the volume of material can between 1 ml and 30 ml; between 2 ml and 30 ml; between 3 and 30 ml; between 4 ml and 30 ml; between 5 ml and 30 ml; between 10 ml and 30 ml; between 15 ml and 30 ml; between 20 ml and 30 ml; between 25 ml and 30 ml; between 0.5 ml and 25 ml; between 1 ml and 25 ml; between 2 ml and 25 ml; between 3 and 25 ml; between 4 ml and 25 ml; between 5 ml and 25 ml; between 10 ml and 25 ml; between 15 ml and 25 ml; between 20 ml and 25 ml; between 0.5 ml and 20 ml; between 1 ml and 20 ml; between 2 ml and 20 ml; between 3 and 20 ml; between 4 ml and 20 ml; between 5 ml and 20 ml; between 10 ml and 20 ml; between 15 ml and 20 ml; between 0.5 ml and 15 ml; between 1 ml and 15 ml; between 2 ml and 15 ml; between 3 and 15 ml; between 4 ml and 15 ml; between 5 ml and 15 ml; between 6 ml and 15 ml; between 7 ml and 15 ml; between 8 ml and 15 ml; between 9 ml and 15 ml; between 10 ml and 15 ml; between 11 ml and 15 ml; between 12 ml and 15 ml; between 13 ml and 15 ml; between 14 ml and 15 ml.

The administration interval for the at least one dose can be one that accommodates at least one inspiration and expiration cycle by the subject to whom the material is administered. In certain embodiments, the administration interval can be between 15 seconds and 30 minutes; between 30 second and 30 minutes; between 1 minute and 30 minutes; between 2 minutes and 30 minutes; between 5 minutes and 30 minutes; between 10 minutes and 30 minutes; between 15 minutes and 30 minutes; between 20 minutes and 30 minutes; between 25 minutes and 30 minutes; between 15 seconds and 25 minutes; between 30 seconds and 25 minutes; between 1 minute and 25 minutes; between 2 minutes and 25 minutes; between 5 minutes and 25 minutes; between 10 minutes and 25 minutes; between 15 minutes and 25 minutes; between 20 minutes and 25 minutes; between 15 seconds and 15 minutes; between 30 seconds and 15 minutes; between 1 minute and 15 minutes; between 2 minutes and 15 minutes; between 5 minutes and 15 minutes; between 10 minutes and 15 minutes; between 15 seconds and 10 minutes; between 30 seconds and 10 minutes; between 1 minute and 10 minutes; between 2 minutes and 10 minutes; between 4 minutes and 10 minutes; between 5 minutes and 10 minutes; between 7 minutes and 10 minutes; between 8 minutes and 10 minutes; between 9 minutes and 10 minutes; between 15 seconds and 5 minutes; between 30 seconds and 5 minutes; between 1 minute and 5 minutes; between 2 minutes and 5 minutes; between 4 minutes and 5 minutes.

In certain embodiments, the pharmaceutically acceptable fluid as administered can have a particle size between 0.1 and 20.0 microns mean mass aerodynamic diameter (MMAD). In certain embodiments, the particle size can be between 0.5 and 20.0; between 0.75 and 20.0; between 1.0 and 20.0; between 2.0 and 20.0; between 3.0 and 20.0; between 4.0 and 20.0; between 5.0 and 20.0; between 7.0 and 20.0; between 10.0 and 20.0; between 12.0 and 20.0; between 15.0 and 20.0; between 16.0 and 20.0; between 17.0 and 20.0; between 18.0 and 20.0; between 0.1 and 15.0; between 0.5 and 15.0; between 0.75 and 15.0; between 1.0 and 15.0; between 2.0 and 15.0; between 3.0 and 15.0; between 4.0 and 15.0; between 5.0 and 15.0; between 7.0 and 15.0; between 10.0 and 15.0; between 12.0 and 15.0; between 14.0 and 15.0; between 0.1 and 10.0; between 0.5 and 10.0; between 0.75 and 10.0; between 1.0 and 10.0; between 2.0 and 10.0; between 3.0 and 10.0; between 4.0 and 10.0; between 5.0 and 10.0; between 7.0 and 10.0; between 8.0 and 10.0; between 9.0 and 10.0; between 0.1 and 5.0; between 0.5 and 5.0; between 0.75 and 5.0; between 1.0 and 5.0; between 2.0 and 5.0; between 3.0 and 5.0; between 4.0 and 5.0; between 0.1 and 4.0; between 0.5 and 4.0; between 0.75 and 4.0; between 1.0 and 4.0; between 2.0 and 4.0; between 3.0 and 4.0; between 0.1 and 3.0; between 0.5 and 3.0; between 0.75 and 3.0; between 1.0 and 3.0; between 1.5 and 3.0; between 2.0 and 3.0; between 0.1 and 2.0; between 0.5 and 2.0; between 0.75 and 2.0; between 1.0 and 2.0; between 1.5 and 2.0; between 0.1 and 1.0; between 0.3 and 1.0; between 0.5 and 1.0; between 0.75 and 1.0 microns.

The pharmaceutically acceptable fluid can be introduced into contact with at least one region of the respiratory tract of the patient at a concentration and in an amount sufficient to reduce pathogen load present in the respiratory tract. It is within the purview of this disclosure that the pharmaceutically acceptable fluid can be introduced continually over a defined interval of minutes, hours or even days. In certain embodiments, the pharmaceutically acceptable fluid can be introduced continuously for an interval of at least 24 hours. In patients presenting with respiratory infections, continuous administration can be discontinued upon reduction in pathogen load either as directly measured or indirectly ascertained by improvement in symptoms such as blood oxygen saturation or the like.

It is also within the purview of this disclosure that the pharmaceutically acceptable fluid can be administered in a series of at least two discrete doses introduced at defined intervals. The intervals for dosing, dosing duration, and number of doses administered will be that sufficient to reduce the pathogen load present in the respiratory tract of the patient either as directly measured or indirectly ascertained by improvement in symptoms such as blood oxygen saturation or the like.

Where desired or required, the pharmaceutically acceptable therapeutic inhalation fluid can be formulated as a composition in which the pharmaceutically acceptable acid composition and antimicrobial peptide component can be co-administered as being admixed in a single composition. Where desired or required, it is considered with in the purview of the present disclosure to administer the pharmaceutically acceptable acid composition and the antimicrobial peptide component sequentially as separate forms as by alternating administrations. It has also been unexpectedly discovered that antimicrobial compounds when admixed with the pharmaceutically acceptable acid composition as disclosed herein can maintain stability and activity for at least 30 minutes after admixture. In certain embodiments, the reduction in pathogen load can be a partial or complete reduction in the pathogen count in the respiratory tract of the patient to whom the pharmaceutically acceptable fluid is administered. Where less than complete reduction in respiratory tract pathogen count is achieved, it is believed that respiratory tract pathogen count reduction, in at least some instances can be sufficient to permit the patient's own immune system response to address or overcome the infectious pathogen either alone or with additional supportive or augmented therapy.

Where the pharmaceutically acceptable inhalation fluid is administered in a plurality of discrete doses, it is contemplated that the pharmaceutically acceptable inhalation fluid can be administered, for example, over 2 to 10 doses in a 24-hour period, with 3 to 4 doses being contemplated in certain embodiments. Each dosing interval can be for a period of 1 second to 120 minutes, with administration intervals between 1 and 60 minutes; 1 and 30 minutes; 1 and 20 minutes; 1 and 10 minutes being contemplated in certain embodiments.

In certain embodiments, when the pharmaceutically acceptable inhalation fluid is administered over a dosing interval, it is contemplated the breathing cycles of the subject facilitate that additional subportions of the pharmaceutically acceptable inhalation fluid dose are incrementally introduced into contact with the respiratory tract over the dosing thereby reducing pathogen load with the continuing incremental addition.

Direct measurement of the reduction in pathogen load in the respiratory tract of the patient can be accomplished by any suitable mechanism such as by swabbing, sampling or the like. In certain embodiments it is contemplated that the reduction in pathogen load can be defined as at least 1% reduction of pathogen population in at least one region of the respiratory tract of the patient as measured at a time between 1 minute and 24 hours after commencement of administration. In certain embodiments, the reduction in pathogen load can be at least 10% as measured at a time between 1 minute and 24 hours after commencement of administration; at least 25%; at least 50%; at least 75%.

It is contemplated that the pharmaceutically acceptable inhalation fluid can be administered prophylactically or therapeutically depending on the physiology and health history of the specific patient. A non-limiting example of prophylactic administration can include routine administration of the pharmaceutically acceptable fluid in a suitable dosing regimen to individuals presenting with a chronic condition with increased risk for respiratory tract infection or complications due to a respiratory tract infection. Another non-limiting example of prophylactic administration is administration of one or more doses of the pharmaceutically acceptable fluid as disclosed herein after exposure to a contagious pathogen.

It is contemplated that administration of the pharmaceutically acceptable inhalation fluid can be accomplished by one or more suitable devices including, but not limited to, nebulizers, cool mist vaporizers, positive pressure inhalers, CPAP units and the like. The device employed can be configured with one or more reservoirs to contain the pharmaceutically acceptable inhalation fluid therein. In certain embodiments, it is contemplated that the various components of the pharmaceutically acceptable inhalation fluid can be contained in admixed relationship in a single reservoir. In certain embodiments, it is contemplated that the administration device can be configured with two or more reservoirs as well means to co-administer or sequentially administer the various components as part of the pharmaceutically acceptable inhalation fluid. In one non-limiting example, it is contemplated that an administration device can include two reservoirs, with one reservoir containing the carrier fluid and pharmaceutically acceptable acid and a second reservoir containing the active pharmaceutical ingredient such as one or more anti-microbial peptides.

The pharmaceutically acceptable inhalation fluid can include at least one pharmaceutically acceptable acid compound that is present at a concentration sufficient to provide a fluid pH between 1.5 and 2.5. The pharmaceutically acceptable inhalation fluid can include at least one acid present in a suitable fluid carrier as desired or required. The pharmaceutically acceptable acid that is employed can be one that, in addition to being pharmaceutically acceptable, is effective, tolerable and non-deleterious to the surrounding tissue present in the respiratory tract of the subject being treated. Suitable acid compounds can be selected from the group consisting of Bronsted acids, Lewis acids and mixtures thereof.

As used herein the term “pharmaceutically acceptable” is defined as having suitable pharmacodynamics and pharmacokinetics such that the therapeutic material is active primarily on the surface of the tissue of the respiratory tract with little or no systemic effect. Ideally, the materials employed produce residual products that are recognized by the body as common metabolites that are rapidly absorbed and metabolized. “Effective” as used herein is defined as materials that are to be effective on the targeted pathogen in vivo with the goal of significantly reducing the pathogen load in order to assist and augment the body's natural defenses. “Tolerable” as defined herein is that the material can be tolerated by the patient at the effective therapeutic concentration without undesirable reactions including, but not limited to, irritation, choking, coughing or the like. “Non-deleterious” as used herein is defined as the material being effective at killing the targeted pathogen with little or no negative effect on the tissue of the respiratory tract of the subject that is in direct contact with the material present at therapeutic concentration levels.

The acid compound employed can be at least one inorganic acid, at least one organic acid or a mixture of at least one inorganic acid and at least one organic acid.

In certain embodiments, pharmaceutically acceptable inhalation fluid will include and can be at least one inorganic acid present in a concentration sufficient to provide a pH at the levels defined herein. Where two or more inorganic acids are employed, the various inorganic acids will present at a ratio sufficient to provide a pH level within the parameters defined in this disclosure. The ratio of respective acids can be modified or altered to meet parameters such as tolerability. Non-limiting examples of suitable inorganic acids include an inorganic acid selected from the group consisting of hydrochloric acid, phosphoric acid, sulfuric acid, hydrobromic acid, phosphoric acid, polyphosphoric acid, hypochlorous acid, and mixtures thereof. In certain embodiments, the pharmaceutically acceptable fluid can include sulfuric acid, hydrochloric acid, hydrobromic acid and mixtures thereof. The present disclosure also contemplates that the at least one inorganic acid in the pharmaceutically acceptable inhalation fluid can be present, in whole or in part, as a salt or salts of the respective inorganic acid. The at least one inorganic acid can be used alone or in combination with other weak or strong organic or inorganic acids or salts thereof in order to obtain the desired pH range.

In certain embodiments, the pharmaceutically acceptable inhalation fluid can include at least one organic acid present in a concentration sufficient to provide a pH at the levels defined herein. In certain embodiments, the at least one organic acid can be present alone or in combination with one or more inorganic acids. Where two or more organic acids are employed, the various organic acids can be present at a ratio sufficient to provide a pH level within the parameters defined in this disclosure. The ratio of respective acids can be modified or altered to meet parameters such as tolerability. Non-limiting examples of organic acids include at least one organic acid selected from the group consisting of acetic acid, trichloroacetic acid, benzenesulfonic acid, citric acid, propionic acid, formic acid, gluconic acid, lactic acid, ascorbic acid, isoascorbic acid, aspartic acid, glutamic acid, glutaric acid and mixtures thereof. In certain embodiments, the organic acid can be at least one of trichloroacetic acid, benzenesulfonic acid, citric acid, propionic acid, formic acid, gluconic acid, lactic acid, ascorbic acid, isoascorbic acid, aspartic acid, glutamic acid, and mixtures thereof.

In certain embodiments, the pharmaceutically acceptable inhalation fluid can include at least one inorganic acid in combination with at least one organic acid listed above. It is also contemplated that the at least one organic acid or the at least one inorganic acid can be present in combination with at least one amino acid. Non-limiting examples of such combination includes for example an amino acid such as aspartic acid or glutamic acid and at least one inorganic acid such as hydrochloric acid, hydrobromic acid, and sulfuric acid required to provide the proper pH range.

Where desired or required, the pharmaceutically acceptable therapeutic inhalation fluid can include a fluid carrier. The fluid carrier component can be a liquid gaseous material suitable for administration to a human, more particularly, the fluid carrier can be one that can be administered as an inhalable or introducible material and come into contact with one or more surfaces present in the at least one region of the respiratory tract of a patient. The fluid carrier component can be a suitable pharmaceutically acceptable protic solvent, a pharmaceutically acceptable aprotic solvent or mixtures thereof. In certain embodiments, the carrier can be a fluid that can be gaseous or can be vaporized, aerosolized or the like by suitable means. Non-limiting examples of suitable carriers include water, organic solvents and the like, present alone or in suitable admixture. Non-limiting examples of organic solvents include materials selected from the group consisting of C2 to C6 alcohols, pharmaceutically acceptable fluorine compounds, pharmaceutically acceptable siloxane compounds, pharmaceutically acceptable hydrocarbons, pharmaceutically acceptable halogenated hydrocarbons and mixtures thereof.

The acid component can be present in an amount sufficient to act on the pathogen present in the respiratory tract of the patient. In certain embodiments, the acid component can be present in an amount up to 10,000 ppm; between 1000 and 10,000 ppm; between 2000 and 10,000 ppm; between 3000 and 10,000 ppm; between 4000 and 10,000 ppm; between 5000 and 10,000 ppm; between 6000 and 10,000 ppm; between 7000 and 10,000 ppm between 8000 and 10,000 ppm; between 9000 and 10,000 ppm. In certain embodiments, the acid component can be present in the pharmaceutically acceptable material solution in an amount between 100 ppm and 2000 ppm; in certain embodiments, the inorganic acid can be present in an amount between 100 ppm and 1700 ppm; between 100 and 1500 ppm; between 100 and 1200 ppm; between 100 and 1000 ppm; between 100 and 900 ppm; between 100 ppm and 800 ppm; between 100 ppm and 700 ppm; and between 100 ppm and 600 ppm. between 500 ppm and 1700 ppm; between 500 and 1500 ppm; between 500 and 1200 ppm; between 500 and 1000 ppm; between 500 and 900 ppm; between 500 ppm and 800 ppm; between 500 ppm and 700 ppm; and between 500 ppm and 600 ppm; between 1000 ppm and 1700 ppm; between 1000 and 1500 ppm; between 1000 and 1200 ppm.

Without being bound to any theory, it is believed acid compound(s) in the pharmaceutically acceptable fluid can function as proton donors which can affect the pathogen(s) present in the at least one region of the respiratory tract of the patient and reduce the pathogen load therein. For example, when sulfuric acid is employed, at least a portion dissociates at low concentration primarily into hydrogen ions and hydrogen sulfate (HSO₄⁻). In its dissociated state sulfuric acid can donate protons to affect pathogens. While this mode of action is mentioned, other modes of action are not precluded by this discussion.

The aforementioned compounds can be present in a suitable liquid material. Non-limiting examples of suitable materials include water of a sufficient purity level to facilitate the availability of the component materials and suitability for end-use applications. In certain embodiments, the water component of the liquid material can be material that is classified as ASTM D1193-06 primary grade. Where desired or required, the water component can be purified by any suitable method, including, but not limited to, distillation, double distillation, deionization, demineralization, reverse osmosis, carbon filtration, ultrafiltration, ultraviolet having a conductivity between 0.05 and 2.00 micro siemens can be employed. It is also within the purview of this disclosure that the water component of the liquid material can be composed of water having a purity greater than primary grade, if desired or required. Water classified as ASTM1193-96 purified, ASTM1193-96 ultrapure or higher can be used is desired or required.

Where desired or required, the composition can also include between 5 and 2000 ppm of pharmaceutically acceptable Group I ions, pharmaceutically acceptable Group II ions and mixtures thereof. In certain embodiments, ions can be selected from the group consisting of calcium, magnesium, strontium and mixtures thereof. In certain embodiments, the concentration of inorganic ion can be between 5 and 900 ppm; between 5 and 800 ppm; between 5 and 700 ppm; between 5 and 600 ppm; between 5 and 500 ppm; between 5 and 400 ppm; between 5 and 300 ppm; 5 and 200 ppm; between 5 and 100 ppm; between 5 and 50 ppm; between 5 and 30 ppm; between 5 and 20 ppm; between 10 and 900 ppm; between 10 and 800 ppm; between 10 and 700 ppm; between 10 and 600 ppm; between 10 and 500 ppm; between 10 and 400 ppm; between 10 and 300 ppm; 10 and 200 ppm; between 10 and 100 ppm; between 10 and 50 ppm; between 10 and 30 ppm; between 100 and 900 ppm; between 100 and 800 ppm; between 100 and 700 ppm; between 100 and 600 ppm; between 100 and 500 ppm; between 100 and 400 ppm; between 100 and 300 ppm; between 200 and 900 ppm; between 200 and 800 ppm; between 200 and 700 ppm; between 200 and 600 ppm; between 200 and 500 ppm; between 200 and 400 ppm; between 200 and 300 ppm; between 300 and 900 ppm; between 300 and 800 ppm; between 300 and 700 ppm; between 300 and 600 ppm; between 300 and 500 ppm; between 300 and 400 ppm. In certain embodiments, the calcium ions can be present as Ca²⁺, CaSO₄⁻¹, and mixtures thereof.

It is contemplated that the acid compound or compounds that is admixed can be produced by any suitable means that results in a material that has limited to no harmful interaction when introduced into contact with at least one region present in the respiratory tract of the patient.

The pharmaceutically acceptable fluid can also include at least one active pharmaceutical ingredient present in suitable therapeutic concentrations. Suitable active pharmaceutical ingredients can be those that have activity that is localized to the region of the respiratory tract to which it is brought into contact. It is also within the purview of this disclosure that suitable active pharmaceutical ingredients can be those which have effect on the larger respiratory system and/or the general systemic effect on the patient. In certain embodiments, the active pharmaceutical ingredient(s) employed can be those which can be administered through the pulmonary system by inhalation or the like. In certain embodiments, it is contemplated that the active pharmaceutical ingredient can be administered as part of a usage or treatment regimen using administration methods other than other than inhalation such as orally or intravenously.

As used herein “Active Pharmaceutical Ingredient” can also include “derivatives” of an Active Pharmaceutical Ingredient, such as, pharmaceutically acceptable salts, solvates, complexes, polymorphs, prodrugs, stereoisomers, geometric isomers, tautomers, active metabolites and the like. Preferably, derivatives include prodrugs and active metabolites. Furthermore, the various “Active Pharmaceutical Ingredients and derivatives thereof” are described in various literature articles, patents and published patent applications and are well known to a person skilled in the art.

In certain embodiments, the at least one active pharmaceutical ingredient can include one or more suitable compounds from classes such as antimicrobials such as antivirals or antibiotics, adrenergic β₂ receptor agonists, steroids, non-steroidal anti-inflammatory compounds, muscarinic antagonists, and the like. In certain embodiments, the pharmaceutically acceptable fluid as disclosed herein can include antiviral compounds with specific or general efficacy against coronaviruses, influenza, and the like to address and treat specific pathogenic infections. Nonlimiting examples of antiviral active pharmaceutical ingredient(s) include one or more compounds selected from the group consisting of amantadine, Lopinavir, linebacker and equivir, Arbidol, a nanoviricide, remdesivir, favipiravir, oseltamivir ribavirin, molnupiravir, Paxlovid, and derivatives and prodrugs thereof as well as combinations of the foregoing. In certain situations, the antiviral active pharmaceutical ingredient(s) can be present in the form that will permit administration via inhalation or other suitable administration into direct or immediate contact with at least a portion of the respiratory tract of the patient. Without being bound to any theory it is believed that the materials such as molnupiravir may be present as a prodrug that could be converted by esterases in the lung to its active metabolite. Combination with the pharmaceutically acceptable fluid administered into contact with the at least one portion of the respiratory tract of the patient in need thereof thereby enhancing bioavailability and/or eliminating one or more side effects of the material administered by other methods.

It is also contemplated that, where desired or required, the antiviral drug can be administered as part of a use or treatment regimen. Orally or intravenously administered antivirals such as neuraminidase inhibitors, Cap-dependent endonuclease inhibitors and the like can be included in a use or treatment regimen.

In certain embodiments, the pharmaceutically acceptable fluid as disclosed herein can include antiviral compounds with specific or general efficacy against coronaviruses, influenza, and the like to address and treat specific pathogenic infections. Non-limiting examples of such antiviral compounds include remdesivir, molnupiravir and the like. The present disclosure contemplates the use of such materials in suitable combination with the pharmaceutically acceptable fluid disclosed herein used prophylactically either upon exposure or routinely, as with at-risk patient populations such as those with chronic illnesses or recognized co-morbidities. The present disclosure also contemplates administration or use of such materials in suitable combination with the pharmaceutically acceptable fluid disclosed hereinafter confirmed diagnosis to symptomatic or asymptomatic individuals. Without being bound to any theory, it is believed that the treatment with or use of the combination as disclosed can provide an effective therapy regimen to address respiratory illnesses including but not limited to SARS-CoV-2, influenza, and the like.

In certain embodiments, the pharmaceutically acceptable fluid can include at least one adrenergic β₂ receptor agonist active pharmaceutical ingredient. Suitable adrenergic β₂ receptor agonists can be those that can be administered by inhalation or other methods of introduction into contact with at least one region of the respiratory tract of the patient. Without being bound to any theory, it is believed that the adrenergic β₂ receptor agonists that are employed can act to cause localized smooth muscle dilation that can result in dilation of bronchial passages. Non-limiting examples of adrenergic β₂ receptor agonist that can be employed in the pharmaceutically acceptable fluid as disclosed herein can include those selected from the group consisting of bitolterol, fenoterol, isoprenaline, levosalbutamol, orciprenaline, pirbuterol, procaterol, ritodrine, salbutamol, terbutaline, albuterol, arformoterol, bambuterol, clenbuterol, formoterol, salmeterol, abediterol, carmoterol, indacaterol, olodaterol, vilanterol, isoxsuprine, mabuterol, zilpaterol, and mixtures thereof.

It is contemplated that, in certain situations, the adrenergic β₂ receptor agonist can be administered in a composition in combination with the pharmaceutically acceptable fluid. It is also contemplated the adrenergic β₂ receptor agonist can be co-administered with the with the pharmaceutically acceptable fluid disclosed herein.

In certain embodiments, the pharmaceutically acceptable fluid can include at least one steroid medication selected from the group consisting of compounds such as beclomethasone, budesonide, ciclesonide, flunisolide, fluticasone, mometasone, and combinations thereof. It is contemplated that, in certain situations, the steroid can be administered in a composition in combination with the pharmaceutically acceptable fluid. It is also contemplated the steroid can be co-administered with the pharmaceutically acceptable fluid disclosed herein.

In certain embodiments, the pharmaceutically acceptable fluid can include at least one inhalable non-steroidal medication such as those selected from the group consisting of compounds such as metabisulphite, adenosine, L-aspirin, indomethacin and combinations thereof.

It is contemplated that, in certain situations, the non-steroidal medication can be administered in a composition in combination with the pharmaceutically acceptable fluid. It is also contemplated the non-steroidal medication can be co-administered with the pharmaceutically acceptable fluid disclosed herein.

In certain embodiments, muscarinic antagonists can be one or more compounds selected from the group consisting of atropine, scopolamine, glycopyrrolate, and ipratropium bromide and the like.

The present disclosure also contemplates the use of one or more antimicrobial peptides (AMPs). “Antimicrobial peptide” as that term is used herein are a class of small molecule peptides generally having between 10 and 80 amino acids that include two or more positively charged residues arginine, lysine, histidine or the like together with a large proportion of hydrophobic residues. The AMP compounds employed in the composition as disclosed herein can include both precursors and as well as active forms as desired or required.

Suitable antimicrobial peptides that can be employed in the disclosure herein include peptides that can be derived from various microorganisms, plants, insects, animals as well as humans.

Nonlimiting examples of AMPs derived from microorganisms include materials from the class Bacterlocin and the class Defensin. Among these are Bacterlocin class AMPs such as those derived from Bacillus spp such as mersacidin; those derived from Lactobacillus gasseri such as lactocillin; materials derived from Lactococcus lactis such as nisin; material derived from Bacillus subtilis such as ericin. Among the Defensin class of AMPs are materials derived from fungi such as Penicillium chrysogenum (PAF) and Aspergillus giganteus (AFP).

Nonlimiting examples of AMPs derived from plants include materials in classes such as Defensin, Thionin and Snakin. Non-limiting examples of Defensin class materials derived from Phaseolus vulgaris (PvD1) and Persea americana (PaDef). Non-limiting examples of Thionin class materials include AMPs derived from Triticum aestivum (α1-purothionin). Non-limiting examples Snakin-class materials include AMPs derived from Ziziphus jujuba (Snakin-Z).

Non-limiting examples of AMPs derived from insects include materials from the classes Cecropin, Defensin, Attacin as well as proline-rich or glycine-rich AMPs. Cecropin-class materials can include AMPs derived from Hyalophora cecropia (DedA); Spodotera litura (Spodopsin Ia). Defensin-class materials can include AMPs derived from Drosophila melanogaster (Drosomycin). Attacin-class materials can include AMPs derived from Hyphantria cunea (Attacin-B). Proline-rich AMPS can include materials derived from Apis mellifera (Abaecin) and glycine-rich AMPs can include material derived from Drosophila melanogaster (Diptericin).

Non-limiting examples of AMPs derived from animals include Cathelicidin-class materials such as BMAP-28 or Protegrin-1 and Brevinin-class materials such as Brevinin-1BYa.

Non-limiting examples of AMPs derived from humans include materials such as hCAP18/LL-37, LL-37, hBD1, hBD2, hBD3, or Histatin-1.

Non-limiting examples of suitable antimicrobial peptides include cathelicidin antimicrobial peptides such as LL-37 and BMAP-28, defensin antimicrobial peptides, transferrin proteins or peptides, non-ribosomal peptides such as gramicidin, lipopeptides such as daptomycin, histatins such as histatin-5 and histatin-1, growth factors such as cytokines and metabologens such as bone morphogenic proteins such as BMAP-1, phospholipid activators such platelet activating factors such as antifungal protein PAF, synthetic cationic peptides such as plexiganan and polycyclic peptide antibiotics such as lantibiotics such as mersacidin.

Non-limiting examples of active forms of antimicrobial peptides and proteins include LL-37, lactoferrin, nisin, subtilin, gramicidin, melittin, histatin such as histatin 1, bone morphogenetic proteins such as bone morphogenic protein-28.

The method as disclosed herein can be employed as a stand-alone treatment regimen or can be employed in combination with other therapy regimens suitable to address and treat the specific respiratory infection. The method can also be used alone or in combination with one or more procedures that can be employed prophylactically to reduce or minimize the risk or symptoms for individuals subsequent to exposure but prior to the onset of symptoms. It is also contemplated that the method as disclosed herein can be employed as a stand-alone treatment regimen for use for individuals at risk for complications or sub-optimal outcomes from respiratory infections. Non-limiting examples of such individuals include those with compromised immune systems, compromised pulmonary function, cardiac challenges, as well as co-morbidities such as age, body weight (obesity) and the like.

The method as disclosed herein can also include the step of administering a composition comprising hypochlorous acid, hydrogen peroxide and mixtures thereof into contact with the at least one region the respiratory tract of the patient. The administration of hypochlorous acid, hydrogen peroxide and mixtures thereof can occur prior to or contemporaneous with the step in which at least one dose of a pharmaceutically acceptable fluid is brought into contact with the at least one region of the respiratory tract of the patient. In certain embodiments, it is contemplated that the composition comprising hypochlorous acid, hydrogen peroxide and mixtures thereof can be co-administered with the pharmaceutically acceptable fluid material as disclosed herein. Where desired or required, the composition comprising hypochlorous acid, hydrogen peroxide and mixtures thereof as dispersed can be configured or sized to contact the same region of the respiratory tract as the pharmaceutically acceptable fluid material or different region.

Where desired or required pharmaceutically acceptable fluid material can be nebulized, aerosolized, or made into a particulate to facilitate administration. Administration of fluid material can be accomplished by direct application as swabbing, spraying, rinsing, emersion, and the like. It is also contemplated that aerosolized or nebulized material can be administered by inhalation if desired or required.

Where the various materials that constitute the pharmaceutically acceptable fluid are aerosolized or nebulized, the pharmaceutically acceptable fluid material(s) can be processed into droplets having a size suitable for inhalation uptake. Non-limiting examples of suitable droplet size include droplets having sizes between 0.1 and 20 μm; between 0.1 and 18 μm; between 0.1 and 17 μm; between 0.1 and 16 μm; between 0.1 and 15 μm; between 0.1 and 14 μm; between 0.1 and 13 μm; between 0.1 and 12 μm; between 0.1 and 12 μm; between 0.1 and 11 μm; between 0.1 and 10 μm; between 0.1 and 9 μm; between 0.1 and 8 μm; between 0.1 and 7 μm; between 0.1 and 6 μm; between 0.1 and 5 μm; between 0.1 and 4 μm; between 0.1 and 3 μm; between 0.1 and 2 μm; between 0.1 and 1 μm; between 0.1 and 0.5 μm; 0.5 and 20 μm; between 0.5 and 18 μm; between 0.5 and 17 μm; between 0.5 and 16 μm; between 0.5 and 15 μm; between 0.5 and 14 μm; between 0.5 and 13 μm; between 0.5 and 12 μm; between 0.5 and 12 μm; between 0.5 and 11 μm; between 0.5 and 10 μm; between 0.5 and 9 μm; between 0.5 and 8 μm; between 0.5 and 7 μm; between 0.5 and 6 μm; between 0.5 and 5 μm; between 0.5 and 4 μm; between 0.5 and 3 μm; between 0.5 and 2 μm; between 0.5 and 1 μm; between 1 and 20 μm; between 1 and 18 μm; between 1 and 17 μm; between 1 and 16 μm; between 1 and 15 μm; between 1 and 14 μm; between 1 and 13 μm; between 1 and 12 μm; between 1 and 11 μm; between 1 and 10 μm; between 1 and 9 μm; between 1 and 8 μm; between 1 and 7 μm; between 1 and 6 μm; between 1 and 5 μm; between 1 and 4 μm; between 1 and 3 μm; between 1 and 2 μm; between 2 and 20 μm; between 2 and 18 μm; between 2 and 17 μm; between 2 and 16 μm; between 2 and 15 μm; between 2 and 14 μm; between 2 and 13 μm; between 2 and 12 μm; between 2 and 11 μm; between 2 and 10 μm; between 2 and 9 μm; between 2 and 8 μm; between 2 and 7 μm; between 2 and 6 μm; between 2 and 5 μm; between 2 and 4 μm; between 2 and 3 μm.

Where desired or required, the acid compound(s) employed can be selected based on the pharmacodynamics and/or pharmacokinetics of the acid compound(s). In certain embodiments of the low pH antimicrobial inhalant making up the pharmaceutically acceptable fluid material can include a dilute sulfuric acid formulation due to its desirable pharmacodynamics and pharmacokinetics. It is believed that the sulfuric acid material will undergo a redox reaction to generate protons (H+) to be absorbed in the mucosa while the sulfate anions will be non-specifically biodistributed into the surrounding tissue for immediate clearance. Unless exposure is excessive, the anion distribution to the body's electrolyte pool is believed to be negligible. Without being bound to any theory, it is believed that the effects of sulfuric acid are the result of the H+ ion (local deposition of H+, pH change) rather than an effect of the sulfate ion. Sulfuric acid per se is not expected to be absorbed or distributed throughout the body. The acid will rapidly dissociate, and the anion will enter the body electrolyte pool, and will not play a specific toxicological role. (See OECD SIDS Sulfuric Acid, 2001, UNEP Publications, p 102). As result little or no systemic effect is expected from dilute inhaled sulfuric acid aerosol, and the only effect will be local to the surfaces of the respiratory system.

The local effect of the released protons can inactivate viruses and other pathogens targeting the mucosal lining of the pulmonary epithelium and endothelium. Dilute sulfuric acid at the therapeutic concentration (˜1.7 pH) provides efficacy at inactivating and/or reducing concentration of human coronavirus within 1 minute based on in vitro suspension tests.

At the proposed exposure concentrations, the resulting proton levels have not demonstrated toxicity on human cells and pulmonary vasculature, likely due to a highly buffered tissue microenvironment that is robust to this short-term change in interspatial pH. This has been shown by acute tissue toxicity and cytotoxicity studies performed within Good Laboratory Practice (GLP) guidelines.

Inhaled inorganic acids such as sulfuric acid at the concentrations contemplated in the present disclosure rapidly dissociate within the proximal pulmonary architecture, absorbing the sulfate ions into the bloodstream. Dahl studied the absorption of ³⁵S radiolabeled sulfuric acid in rats, guinea pigs, and dogs, revealing that rat and guinea pig animal models have very similar PK/PD parameters with 170 and 230 second ³⁵S half-lives. The half-life of the ³⁵S radiolabeled sulfuric acid in the dog studies varied significantly depending on the specific respiratory system administration site. Deep-lung sulfuric acid administration demonstrated a 2-3 minute half-life similar to the rats and guinea pigs. The half-life was significantly longer for administration to higher regions within the bronchi and sinus cavities. (see Dahl, Clearance of Sulfuric Acid-Introduced ³⁵S from the Respiratory Tracks of Rats, Guinea Pigs and Dogs Following Inhalation or Instillation, Fundamental and Applied Toxicology 3:293-297 (1983)).

The therapeutic inhalant demonstrates anti-viral therapeutic potential in the peripheral lung tissues with a half-life of ˜2-3 minutes until absorption. Although sulfuric acid neutralization was not directly measured within the respiratory system, previous in vitro studies predict virus, bacteria, and fungi replication inhibition within 1 minute.

Also disclosed herein is a kit for use in the treatment or prevention of a respiratory illness that includes at least one container for administering the pharmaceutically acceptable fluid into the respiratory tract of a patient in need thereof that is connectable to a respiratory delivery device having at least one chamber. The at least one chamber contains at least one dose of a pharmaceutically acceptable fluid as disclosed herein. The pharmaceutically acceptable fluid includes a liquid carrier and at least one acid compound, wherein the pharmaceutically acceptable fluid has a pH less than 2.5 and a container for administering the pharmaceutically acceptable fluid into the respiratory tract of a patient in need thereof.

The kit can also include means for administering the pharmaceutically acceptable fluid to at least a portion of the respiratory tract of the patient in need thereof. Non-limiting examples of suitable means for administering the pharmaceutically acceptable fluid to at least a portion of the respiratory tract of the patient in need thereof can include devices like inhalers, metered dose inhalers, nebulizers such as PARI nebulizers and the like. The administering means can include at least one mechanism that delivers the fluid in a vaporized, atomized, or nebulized state. “Nebulizer” as the term is used herein is a drug delivery device used to administer medication in a form that can be inhaled into the lungs using oxygen, compressed air, ultrasonic power, or the like to break up solutions into small aerosol droplets. Non-limiting examples of nebulizers that can be used to dispense the pharmaceutically acceptable fluid as disclosed herein can be a jet nebulizer, a soft mist inhaler, an ultrasonic nebulizer, or the like. PARI nebulizers are commercially available PARI Respiratory Equipment, Inc., Midlothian VA.

The kit can also include a suitable mask or oral insert to direct material into the oral and/or nasal cavity of the patient.

Also disclosed is a respiratory inhalant device that includes a reservoir having at least one interior chamber and a dispenser in fluid communication with the reservoir. The container includes pharmaceutically acceptable fluid as disclosed herein contained in the at least one interior chamber.

The respiratory inhalant device also includes a dispenser in fluid communication with the reservoir that is configured to dispense a measured portion of the pharmaceutically acceptable fluid from the reservoir into inhalable contact with at least one portion of a respiratory tract of a patient having a respiratory illness. The pharmaceutically acceptable fluid dispensed in a droplet size between 0.5 and 5.0 microns mean mass diameter. In certain embodiments, the dispenser can include suitable tubing and an outlet member. The outlet member can be configured as a mask that can be removably fitted to the patient or a pipe-like member that can be removably inserted into the mouth of the patient, in certain embodiments. Other delivery members may include nasal cannulae, or the like.

The respiratory illness can be at least one of a viral pathogen, a bacterial pathogen, a fungal pathogen such as a viral pathogen such as one of coronavirus, an influenza virus, a parainfluenza virus, respiratory syncytial virus, a rhinovirus. In certain embodiments, the viral pathogen can be a beta coronavirus selected from the group consisting of SARS-CoV, SARS-CoV-2, MERS-CoV, and mixtures thereof.

Also disclosed herein is a system for treating a respiratory infection caused by at least one antimicrobial-resistant pathogen that includes a medication delivery device with at least one medication storage chamber and a medication outlet member in fluid communication with the medication chamber. The at least one medication storage chamber contains a pharmaceutically acceptable therapeutic inhalation fluid composition that comprises a fluid carrier; and a pharmaceutically acceptable acid composition wherein the pharmaceutically acceptable acid composition is present in the carrier in an amount sufficient to provide a pH between 1.5 and 2.5 and at least one antimicrobial peptide. The system is configured such as that at least a portion of the pharmaceutically acceptable therapeutic inhalation fluid composition is dispatched through the medication outlet member in a vaporized or atomized state. Where desired or required, the particles can be of sizes as discussed previously. Non-limiting examples of suitable devices that can be employed in the system disclosed are nebulizers, vaporizers and the like.

Where desired or required, the pharmaceutically acceptable acid composition can be as disclosed previously. Where desired or required, the at least one antimicrobial peptide can be one or more of the compounds discussed previously. In certain embodiments, the pharmaceutically acceptable therapeutic inhalation fluid composition can consist of a fluid carrier a pharmaceutically acceptable acid composition wherein the pharmaceutically acceptable acid composition is present in the carrier in an amount sufficient to provide a pH between 1.5 and 2.5 and at least one antimicrobial peptide.

In certain embodiments, the medication delivery device can include at least two medication chambers in which a first medication chamber contains a composition that comprises a fluid carrier and a pharmaceutically acceptable acid composition wherein the pharmaceutically acceptable acid composition is present in the carrier in an amount sufficient to provide a pH between 1.5 and 2.5. The second chamber can contain a composition comprising at least one antimicrobial peptide. The system can include at least one mixing apparatus in communication with the first medication chamber and the second medication chamber with the mixing apparatus communicating with the medication outlet. In certain embodiments, the pharmaceutically acceptable acid composition can be maintained in one first medication chamber and the at least one antimicrobial peptide can be maintained in a second medication chamber.

It is also contemplated that the that present disclosure is directed to a kit for use in the treatment or prevention of a respiratory illness. The kit comprises a container connectable to a respiratory delivery device for administering the pharmaceutically acceptable fluid into the respiratory tract of a patient in need thereof, the container having at least one chamber, the chamber containing at least one dose of a pharmaceutically acceptable fluid which comprises a liquid carrier and at least one acid compound, wherein the pharmaceutically acceptable fluid has a pH less than 2.5 and at least one antimicrobial peptide; and at least one device for conveying the pharmaceutically acceptable fluid from the container into the respiratory tract of a patient in need thereof. Various embodiments of the pharmaceutically acceptable inhalation fluid have been discussed in the present disclosure. The kit can include means for administering the pharmaceutically acceptable inhalation fluid into contact with at least a portion of the respiratory tract of a subject and can include at least one mechanism that delivers the fluid in a vaporized, atomized or nebulized state.

Where desired or required, the disclosure also contemplates a respiratory inhalant device that includes a reservoir having at least one interior chamber and a pharmaceutically acceptable inhalation fluid contained in the interior chamber. The pharmaceutically acceptable inhalation fluid is composed of an acid compound, the acid compound selected from the group consisting of at least one organic acid, at least one inorganic acid, and mixtures thereof and at least one antimicrobial peptide in a carrier such as a fluid carrier at a pH less than 2.5 as disclosed and discussed previously in this Specification. The devise also includes a dispenser in fluid communication with the reservoir that is configured to dispense a measured portion of the pharmaceutically acceptable fluid from the reservoir into inhalable contact with at least one portion of a respiratory tract of a patient having a respiratory illness, the pharmaceutically acceptable fluid in at a droplet size, for example between 0.5 and 5.0 microns mean mass diameter. The illness to be treated can be one an acute respiratory illness caused by at least one of an antimicrobial resistant viral pathogen, an antimicrobial resistant bacterial pathogen, an antimicrobial resistant fungal pathogen as discussed previously.

In order to further illustrate the present disclosure, the following examples are presented. The Examples are for illustration purposes and are not to be considered limitative of the present disclosure.

### Examples 1-20

Purpose: A modified cytotoxicity protocol was developed focusing on the cytotoxicity of the residual anions of the acid formulations. Twenty different acids formulations were tested, each in the range of 1.95 to 1.5 pH. Each formulation was diluted with distilled water to hydronium concentrations of 11.22 mM (pH 1.95), 14.25 mM (pH 1.85), 19.95 mM (pH 1.70), 25.12 mM (pH 1.60), and 31.62 mM (pH 1.50). It was then titrated with NaOH to 7.4 pH and applied to HepG2 (human liver) cell culture in triplicate replicates.

Results: The study results are shown in Table 1.

TABLE 1
Cell Viability of Residual Anions from Neutralized Acids

| | | Passed?, | Passed?, | Passed?, | Passed?, | Passed?, |
|---|---|---|---|---|---|---|
| | | %Viability | % Viability | % Viability | % Viability | % Viability |
| | Sample | @ pH 1.5 | @ pH 1.6 | @ pH 1.7 | @ pH 1.85 | @ pH 1.9 |
| 1 | HCl | Yes, 130% | Yes, 125% | Yes, 126% | Yes, 141% | Yes, 102% |
| 2 | HCl + aspartic | Yes, 101% | Yes, 118% | Yes, 114% | Yes, 128% | Yes, 128% |
| 3 | Sulfuric | Yes, 117% | Yes, 130% | Yes, 121% | Yes, 133% | Yes, 140% |
| 4 | Sulfuric + | Yes, 92.0% | Yes, 150% | Yes, 133% | Yes, 122% | Yes, 128% |
| | Albuterol | | | | | |
| 5 | Sulfuric + | Yes, 55.5% | Yes, 62.1% | Yes, 69.7% | Yes, 70.8% | Yes, 82.7% |
| | Adenosine | | | | | |
| 6 | HBr | Yes, 69.1% | Yes, 77.3% | Yes, 75.3% | Yes, 81.7% | Yes, 110% |
| 7 | Citric + HCl | Yes, 55.8% | Yes, 62.1% | No, 47.2% | No, 45.5% | No, 43.9% |
| 8 | Hydroxyacetic +. | Yes, 67.7% | Yes, 76.3% | Yes, 78.3% | Yes, 65.7% | Yes, 72.7% |
| | Sulfuric | | | | | |
| 9 | Trichloroacetic | No, 34.1% | Yes, 60.2% | Yes, 52.9% | Yes, 70.7% | Yes, 97.3% |
| 10 | Trifluoroacetic | Yes, 63.2% | Yes, 63.4% | Yes, 69.5% | Yes, 78.8% | Yes, 122% |
| 11 | Benzene sulfonic | Yes, 56.5% | Yes, 61.0% | Yes 68.0% | Yes, 83.9% | Yes, 114% |
| 12 | Sulfuric + | No, 2.29% | No, 2.56% | No, 1.10% | No, 4.36% | No, 1.23% |
| | K sorbate | | | | | |
| 13 | Monochloroacetic | No, 3.17% | No, 5.24% | No, 5.29 | No, 2.73% | No, 1.01% |
| 14 | Phosphoric | No, 1.15% | No, 2.03% | No, 4.23% | Yes, 75.9% | No, 5.02% |
| 15 | Hydroxyacetic + | No, 44.5% | Yes, 61.0% | Yes, 54.5% | Yes, 60.2% | Yes, 117% |
| | HCl | | | | | |
| 16 | Lactic | Yes, 52.0% | Yes, 50.3% | Yes, 70.6% | Yes, 72.8% | Yes, 56.1% |
| 17 | Isoacorbic + | No, 5.87% | No, 17.0% | No, 3.83% | No, 39.5% | Yes, 89.3% |
| | sulfuric | | | | | |
| 18 | Isoascorbic + HCl | Yes, 79.3% | Yes, 102% | Yes, 116% | Yes, 119% | Yes, 104% |
| 19 | Adenosine, HCl | No, 44.0% | No, 40.1% | No, 44.0% | No, 48.9% | Yes, 50.7% |
| | and aspartic | | | | | |
| 20 | Aspartic + sulfuric | No, 43.2% | Yes, 52.0% | Yes, 68.3% | Yes, 67.2% | Yes, 86.6% |

Note:
If viability was less than 50% it was flagged as NO PASS indicated as “No”. Considered to be cytotoxic and/or hepatoxic

Conclusions: The cytotoxicity of residual anions from acid formulations varies significantly. Based on this modified cytotoxicity testing the least toxic acid formulations were sulfuric (example 3), hydrochloric (example 1), hydrochloric+aspartic (example 2), sulfuric+albuterol (4), and isoascorbic+hydrochloric (example 18). Hydrobromic would be less desirable (example 6). Phosphoric was generally cytotoxic (example 14). The formulations with sulfuric acid and/or hydrochloric acid are preferred embodiments.

Many organic acids are too weak and when used alone cannot meet the 2.5 or lower pH requirement. Several of the stronger inorganic acids when used in the concentration of below 2.5 pH have unacceptable cytotoxicity or hepatoxicity.

Another preferred embodiment is to include a small amount of organic acid to an inorganic acid mixture such as hydrochloric+aspartic (example 2) and isoascorbic+hydrochloric (example 18). Adding a small amount of organic acid to the inorganic acid formulation may improve wetting, bacterial cell penetration and/or provide antioxidation benefits over the inorganic acid mixture alone.

### Formulation

In certain embodiments, pharmaceutically acceptable fluid will include at least one inorganic acid present in a concentration sufficient to provide a pH at the levels defined herein. When two or more inorganic acids, two or more organic acids, or when a mixture of inorganic and organic acids are employed, the various acids will present at a ratio sufficient to provide a pH level within the parameters defined in this disclosure. The ratio of respective acids can be modified or altered to meet parameters such as tolerability. Non-limiting examples of suitable inorganic acids include an inorganic acid selected from the group consisting of hydrochloric acid, phosphoric acid, sulfuric acid, hydrobromic acid, phosphoric acid, polyphosphoric acid, hypochlorous acid, and mixtures thereof. In certain embodiments, the pharmaceutically acceptable fluid can include sulfuric acid, hydrochloric acid, hydrobromic acid and mixtures thereof.

The present disclosure also contemplates that at least one inorganic acid in the pharmaceutically acceptable fluid can be present in whole or in part as a salt or salts of the respective inorganic acid. At least one inorganic acid can be used alone or in combination with other weak or strong organic or inorganic acids or salts thereof to obtain the desired pH range.

In certain embodiments, the pharmaceutically acceptable fluid can include at least one organic acid present in a concentration sufficient to provide a pH at the levels defined herein. In certain embodiments, at least one organic acid can be present in combination with one or more inorganic acids. Most organic acids cannot achieve the therapeutic pH range without formulating with strong inorganic acids. Mixing one or more organic acid in a formulation of one or more inorganic acids can improve the low pH therapeutic by improving wetting, increasing bacterial cell wall penetration, providing antioxidant properties or other improvements. Non-limiting examples of organic acids include at least one organic acid selected from the group consisting of acetic acid, trichloroacetic acid, benzenesulfonic acid, propionic acid, formic acid, gluconic acid, lactic acid, ascorbic acid, isoascorbic acid, aspartic acid, glutamic acid, glutaric acid and mixtures thereof,

In certain embodiments, the organic acid can be at least one of trichloroacetic acid, benzenesulfonic acid, propionic acid, formic acid, gluconic acid, lactic acid, ascorbic acid, isoascorbic acid, aspartic acid, glutamic acid, and mixtures thereof.

In certain embodiments, the method and/or composition as disclosed herein can have a pH less than 2.5; less than 2.4; less than 2.3; less than 2.2, less than 2.1; less than 2.0; less than 1.95; less than 1.9, less than 1.8; less than 1.7; less than 1.6; less than 1.5; less than 1.0 with lower ranges being determined by the sinus and lung condition and health of the patient. In certain embodiments, the composition can have a have a pH between 1.4 and 1.95 between 1.5 and 1.95; between 1.6 and 1.95; between 1.7 and 1.95; between 1.8 and 1.95; between 1.9 and 1.95; between 1.4 and 1.9; between 1.5 and 1.9; between 1.6 and 1.9; between 1.7 and 1.9; between 1.8 and 1.9; between 1.4 and 1.8; between 1.5 and 1.8; between 1.6 and 1.8, between 1.7 and 1.8; between 1.4 and 1.7; between 1.5 and 1.7, between 1.6 and 1.7; between 1.4 and 1.6; between 1.5 and 1.6; between 1.4 and 1.5.

The aforementioned compounds can be present in a suitable liquid material. Non-limiting examples of suitable materials include water of a sufficient purity level to facilitate the availability of the component materials and suitability for end-use applications. In certain embodiments, the water component of the liquid material can be a material that is classified as ASTM D1193-06 primary grade. Where desired or required, the water component can be purified by any suitable method, including, but not limited to, distillation, double distillation, deionization, demineralization, reverse osmosis, carbon filtration, ultrafiltration, ultraviolet having a conductivity between 0.05 and 2.00 micro siemens can be employed. It is also within the purview of this disclosure that the water component of the liquid material can be composed of water having a purity greater than primary grade, if desired or required. Water classified as ASTM1 193-96 purified, ASTM1 193-96 ultrapure or higher can be used if desired or required,

Where desired or required, the composition can also include between 5 and 2000 ppm of pharmaceutically acceptable Group I ions, pharmaceutically acceptable Group II ions and mixtures thereof. In certain embodiments, ions can be selected from the group consisting of calcium, magnesium, strontium, and mixtures thereof. In certain embodiments, the calcium salts may be incorporated such as Calcium sulfate, Calcium acetate, Calcium chloride, etc and mixtures thereof. In certain embodiments, soluble magnesium, strontium and alkali metal ions may also be added.

In certain embodiments, the concentration of inorganic Group I or Group II cations can be between 5 and 900 ppm; between 5 and 800 ppm; between 5 and 700 ppm; between 5 and 600 ppm; between 5 and 500 ppm; between 5 and 400 ppm; between 5 and 300 ppm; 5 and 200 ppm; between 5 and 100 ppm; between 5 and 50 ppm; between 5 and 30 ppm; between 5 and 20 ppm; between 10 and 900 ppm; between 10 and 800 ppm; between 10 and 700 ppm; between 10 and 600 ppm; between 10 and 500 ppm; between 10 and 400 ppm; between 10 and 300 ppm; 10 and 200 ppm; between 10 and 100 ppm; between 10 and 50 ppm; between 10 and 30 ppm; between 100 and 900 ppm; between 100 and 800 ppm; between 100 and 700 ppm; between 100 and 600 ppm; between 100 and 500 ppm; between 100 and 400 ppm; between 100 and 300 ppm; between 200 and 900 ppm; between 200 and 800 ppm; between 200 and 700 ppm; between 200 and 600 ppm; between 200 and 500 ppm; between 200 and 400 ppm; between 200 and 300 ppm; between 300 and 900 ppm; between 300 and 800 ppm; between 300 and 700 ppm; between 300 and 600 ppm; between 300 and 500 ppm; between 300 and 400 ppm.

In certain embodiments, the low pH therapeutic can be formulated with, co-administered with, or used as an adjunct therapy with sinusitis therapies including zinc acetate, zine gluconate and other zinc compounds.

In certain embodiments, the low pH therapeutic can be formulated with, co-administered with, or used as an adjunct therapy with Antimicrobial Peptides for example lactoferrin, defensins and meucins.

In certain embodiments, the low pH therapeutic can be formulated with, co-administered with, or used as an adjunct therapy with antibiotics therapies for sinusitis including Amoxicillin, Azithromycin, Cephalospins, Augmentin, Cipro, Levaquin, Avela, Vancomycin and Aminoglycide.

In certain embodiments, the low pH therapeutic can be formulated with, co-administered with, or used as an adjunct therapy with therapies for sinusitis. The sinusitis therapies include analgesics such as nonsteroidal anti-inflammatory drugs (NSAID) including ibuprofen, acetaminophen and aspirin. These sinusitis therapies include nasal decongestants both oral and intranasal including oxymetazoline. These sinusitis therapies include antihistamines including azelastine. These sinusitis therapies include bronchial dilators including inhaled ipratropium and albuterol, both individually and combined. These sinusitis therapies include corticosteroids include fluticasone, budesonide, azelastine, mometasone, triamcinolone, beclomethasone, ciclesonide. These sinusitis therapies include non-steroidal anti-inflammatory sprays including cromolyn sodium.

### Antimicrobial Resistance

The U.S. Centers for Disease Control and Prevention (CDC) and the World Health Organization (WHO) categorize antimicrobial-resistant (AMR) pathogens as a looming threat to human health. While AMR genes occur naturally in the environment, the use of antibiotics has selected for the presence of AMR genes. The lack of rapid diagnostic methods to identify bacterial pathogens and AMR genes in clinical settings has resulted in the often-unnecessary use of broad-spectrum antibiotics.

In February 2017, to focus and guide research and development related to new antibiotics, the WHO published its list of pathogens for which new antimicrobial development is urgently needed. Within this broad list, ESKAPE (Enterococcus faecium, Staphylococcus aureus, Klebsiella pneumoniae, Acinetobacter baumannii, Pseudomonas aeruginosa, and Enterobacter species) pathogens were designated “priority status”. (De Oliveira, D. et al. (2020). Antimicrobial Resistance of ESKAPE pathogens, Clinical Microbiology Reviews).

The WHO further prioritized three Gram-negative multidrug resistant bacteria as critical that pose a particular threat in hospitals, nursing homes, and among patients whose care requires devices such as ventilators and blood catheters.

WHO Priority 1: CRITICAL Pathogens for New Antibiotics
Acinetobacter baumannii, carbapenem-resistant
Pseudomonas aeruginosa, carbapenem-resistant
Enterobacteriaceae, carbapenem-resistant, extended Spectrum Beta-Lactamase (ESBL). These bacteria cause severe and often deadly infections such as bloodstream infections and pneumonia. These bacteria have become resistant to a large number of antibiotics, including carbapenems and third generation cephalosporins—the best available antibiotics for treating multi-drug resistant bacteria.

It has been estimated that in 2019 4.95 million deaths were associated with bacterial AMR including 1.27 million deaths attributable to AMR. The leading indication of AMR mortality was respiratory infections. (Murray, C. et al. (2022). Global burden of bacterial antimicrobial resistance in 2019: a systematic analysis, Lancet). Therefore, there is an urgent global need for innovative antimicrobial therapies for ESKAPE and other AMR pathogens.

### Low pH Therapeutic Efficacy on ESKAPE Bacteria

A low pH pulmonary therapeutic has been described in U.S. Pat. No. 11,642,372 using an acid formulation with a pH of about 1.72. This formulation demonstrated an excellent safety profile in a Phase I First-in-Human clinical trial when administered with a nebulizer to pulmonary epithelial tissues. This study with 24 COVID-19 PCR positive subjects indicated that there was no treatment related adverse events and patients had a reduction of symptoms from the treatment.

In vitro laboratory tests of similar low pH formulations using a 1-minute suspension tests demonstrated efficacy on a wide range of bacteria (Gram-negative and Gram-positive), viruses (encapsulated including coronaviruses and non-encapsulated) and fungi. These tests also demonstrated efficacy on several drug resistant microorganisms including Pseudomonas aeruginosa. These tests indicate a variety of acid formulations below 2.5 pH demonstrate anti-viral, anti-bacterial and anti-fungal efficacy.

The exact MOA is not known but may be caused by the sudden temporary change in extra-cellular proton concentration from the low pH therapeutic, which disrupts the pH homeostasis of the bacteria and other pathogens while the host eukaryote tissues have been demonstrated to be less sensitive to this transitory effect. One potential reason for this may be that both Gram-positive and Gram-negative bacteria have a negative charge on their cellular membranes, while host tissues have no charge. In an environment of host tissues and bacteria electrostatic forces will attract positively charged protons from the low pH therapeutic to the negative charged bacteria cell walls improving antibacterial efficacy.

### Examples 21-32

Purpose: The low pH therapeutic composed of a dilute solution sulfuric acid was applied to both drug sensitive and drug resistant strains of the six ESKAPE bacteria to determine its efficacy.

Results: The results summarized in Table 2.

TABLE 2
Low pH Therapeutic Efficacy on ESKAPE Pathogen

| | ESKAPE | Gram | Antimicrobial | ATCC | pH As | Eff | |
|---|---|---|---|---|---|---|---|
| | Species | stain | Resistant? | # | Applied | (log) | Eff (%) |
| 21 | Enterococcus | Gram+ | yes | 51559 | 1.66 | 0.16 | 30.59% |
| 22 | faceium | | no | 19434 | 1.66 | 1.51 | 96.91% |
| 23 | Staphylococcus | Gram+ | yes | 33591 | 1.66 | 0.21 | 38.22% |
| 24 | aureus | | no | 25923 | 1.66 | 0.50 | 68.71% |
| 25 | Klebsiella | Gram− | yes | BAA- | 1.66 | >5.92 | >99.99988% |
| | | | | 2146 | | | |
| 26 | pneumoniae | | no | 4352 | 1.66 | >6.59 | >99.99997% |
| 27 | Acinebacter | Gram− | yes | BAA- | 1.66 | 5.11 | 99.9992% |
| | | | | 1605 | | | |
| 28 | baumannii | | no | 19606 | 1.66 | >6.20 | >99.9999% |
| 29 | Pseudomonas | Gram− | yes | BAA- | 1.66 | >6.25 | >99.99994% |
| | | | | 2108 | | | |
| 30 | aeruginosa | | no | 27853 | 1.66 | >6.8 | >99.99998 |
| 31 | Enterobacter | Gram− | yes | BAA- | 1.66 | >6.00 | >99.9999% |
| | | | | 2468 | | | |
| 32 | cloacae | | no | 13047 | 1.66 | 4.11 | 99.992% |

Tested In Accordance with ASTM E2315, 1 minute, no soiling, non-GLP, single-replicant

Conclusions: Efficacy of the low pH therapeutic was 4.11 log (99.992%) or more on all Gram-negative strains (examples 25-32) and most have 6 log efficacies. The four AMR resistant Gram-negative bacteria stains (examples 25, 27, 29, and 31) include bacteria that are carbapenem resistant and are beta-lactamase producers, considered to by the WHO to be the bacteria with the highest priority need for new therapeutics.

The efficacy of the low pH therapeutic on these drug resistant strains of Gram-negative bacteria was as high as on the drug sensitive strains. This indicates that the AMR resistance mechanisms for Gram-negative bacteria do not provide protection to the low pH therapeutic Mechanism of Action (MOA).

Efficacy is lower on Gram-positive bacteria and ranged between 0.16 log (30.59%) and 1.51 log (96.91%) (examples 1-4). For both Gram-positive strains the drug resistant or AMR strains had lower efficacy than the drug sensitive strains. The antimicrobial resistance mechanisms of these two Gram-positive bacteria may reduce efficacy of the low pH therapeutic.

Although efficacy was lower on Gram-positive strains than Gram-negative bacteria strains, the low pH therapeutic still had in vitro efficacy. To achieve significant clinical efficacy on these Gram-positive bacteria the number of low pH therapeutic treatments (#doses) and/or the treatment time may need to be increased.

### Discussion on Bacterial Efficacy

It is unexpected that low pH therapeutics generally have higher efficacy on Gram-negative than Gram-positive bacteria, which enables many novel therapeutic approaches. In addition to conventional treatment of bacterial infections with systemic antibiotics that preferentially target Gram-positive bacteria, the clinician may prefer to treat the bacterial infection using only the low pH therapeutics as disclosed herein that preferably target Gram-negative bacteria or may treat using the low pH therapeutic as an adjunct therapy to traditional antibiotics to eliminate all bacteria.

The difference in cell wall structure and thickness between Gram-positive and Gram-negative bacteria may account for this difference in efficacy of the low pH therapeutic. Both Gram-positive and Gram-negative bacteria have peptidoglycan cell walls providing a rigid exoskeleton, but the thickness of the cell wall is significantly greater in Gram-positive bacteria. Whereas Gram-negative peptidoglycan is only a few nanometers thick, representing one to a few layers, Gram-positive peptidoglycan is normally 30-100 nm thick and contains many layers (Silhavy, T J, et al. (2010). The bacterial cell envelope. Cold Spring Harb Perspect Bio). The thicker peptidoglycan layer in Gram-positive bacteria may provide additional resistance to the low pH MOA.

Gram-negative bacteria have an additional protective Outer Membrane (OM) not found in Gram-positive species. The OM enables additional antibiotic resistance mechanisms such as efflux pumps that can expel antibiotic and other harmful molecules. Due to their distinctive structure, Gram-negative bacteria are more resistant than Gram-positive bacteria, and cause significant morbidity and mortality worldwide (Breiyeh, Z. et al. (2020). Resistance of Gram-Negative Bacterial to Current Antibacterial Agents and Approaches to Resolve It, Molecules).

Like other biological membranes, the OM is a lipid bilayer, but importantly, it is not a phospholipid bilayer. The OM does contain phospholipids; they are confined to the inner leaflet of this membrane. The outer leaflet of the OM is composed of glycolipids, principally lipopolysaccharide (LPS). LPS is an infamous molecule because it is responsible for the endotoxic shock associated with the septicemia caused by Gram-negative organisms. The human innate immune system is sensitized to this molecule because it is a sure indicator of infection.

Polysaccharides can be cleaved with acids such as found in the low pH therapeutic. Based on this potential MOA the low pH therapeutic may not only kill Gram-negative bacteria, it may reduce the endotoxicity from the bacteria and its inflammatory effects. This may provide broad anti-inflammatory benefits.

### Increasing Low pH Therapeutic Efficacy Using Antimicrobial Peptides

In some clinical cases the low pH inhalation therapeutics have may have insufficient efficacy on certain pathogenic bacteria species. This is more likely on harder to kill Gram-positive bacteria including Staphylococcus aureus and mycobacteria including Mycobacterium tuberculosis. It may therefore be desirable to increase the efficacy of low pH therapeutics against these pathogens.

Antimicrobial Peptides (AMPs) are class of small peptides which are part of the innate immune system that provide inhibitory effects against bacteria, fungi, viruses, and parasites. Research has identified more than 2000 peptides and they have been studied extensively for their therapeutic properties. Most of these AMPs are cationic, or positively charged, which improve their efficacy on negative charged bacteria. Two of the AMPs most studied are LL-37 and human lactoferrin. They both naturally exist in the human pulmonary system, but these AMPs have not previously been studied in compositions having a pH less than 3.0 such as low pH therapeutics.

### Examples 33-38

Purpose: Test and compare the efficacy of LL-37 and human lactoferrin in a low pH therapeutic and compare with efficacy in a neutral water solution. Since the reaction speed of the two different MOAs are different, a modified test protocol was developed, which is summarized below.
a. The AMP solutions were prepared immediately prior to use (within 20 minutes) by the test laboratory by mixing the dry AMP with the water or dilute sulfuric acid solution. Heretofore, it has been believed that AMPs are unstable in low pH environments. Mixture of dry powder immediately prior to use is believed to address concerns regarding minimization of in vitro stability.
b. The prepared AMP solution was then mixed with the bacteria containing media. After one minute the acid solution was neutralized. This simulates the short antimicrobial period for the elevated proton concentration in vivo. The bacteria population was measured to determine efficacy after one minute.
c. The AMPs retained in the solution are not expected to stop their antimicrobial activity after acid neutralization, so a second bacteria population measurement was made after 24 hours.

Results: The test results are shown in Table 3.

TABLE 3
Efficacy of Low pH Therapeutic with Selected AMPs on S. aureus

| | | | | 1-min | 24-hour |
|---|---|---|---|---|---|
| | | Antimicrobial | pH As | Efficacy | Efficacy |
| | Solvent | Peptide | Applied | (log) | (log) |
| 33 | Sulfuric Acid | none | 1.48 | 0.37 | 2.88 |
| 34 | Sulfuric Acid | LL-37 | 1.46 | 0.18 | 4.88 |
| 35 | Sulfuric Acid | lactoferrin | 1.50 | 0.02 | 5.49 |
| 36 | Sulfuric Acid | LL-37 + | 1.49 | 0.04 | >6.45 |
| | | lactoferrin | | | |
| 37 | Water* | lactoferrin | 5.10 | 0.05 | 0.16 |
| 38 | Water* | LL-37 | 6.30 | 4.15 | >6.45 |

Tested In Accordance With ASTM E2315, 1 minute contact time, no soiling, non-GLP, single-replicant Bacteria Tested: Staphylococcus aureus ATCC 6538
*Comparative Examples

Conclusions: In neutral water solutions the AMP LL-37 was highly effective (comparative example 38) and the lactoferrin had minimal efficacy (comparative example 37). In a low pH therapeutic solution LL-37, lactoferrin and combined the AMP LL-37 (examples 34-36) all improved efficacy as compared to the low pH therapeutic without AMPs (example 33).

Alaiwa studied the pH effect of several AMPs on the efficacy to kill S. aureus (Alaiwa, M. et al. (2014). pH modulates the activity and synergism of the airway surface liquid antimicrobials B-definsin and LL-37, PNAS). It was determined that reducing the pH from 8.0 to 6.8 reduced the ability of LL-37 and beta-definsin-3 to kill S. aureus. The approach of this study was to compare the antibacterial effect at different constant pH levels over two hours to simulate the effect of slightly lower constant pH found in cystic fibrosis patients. The study concluded that lowering pH generally reduces AMP efficacy.

The in vivo pH level when applying the low pH therapeutic is significantly lower than the pH level tested by Alaiwa, but lasts only for 1 minute or less. LL-37 efficacy between 6.30 pH (example 38) and 1.5 pH (example 34) was decreased. Surprisingly, lactoferrin which is not effective at neutral pH (example 37) demonstrated significant efficacy improvement when combined with the low pH therapeutic (example 35) over the low pH therapeutic alone (example 33). A potential advantage of using Lactoferrin over LL-37, is that Lactoferrin may be more stable in acid solutions and more stable during the nebulization process.

The low pH therapeutic may contain or may be co-administered with one or more Antimicrobial Peptides including lactoferrin, defensins and meucins. Adding one or more AMPs improves efficacy on pathogens which are less sensitive to the low pH therapeutic alone. This would include certain Gram-positive bacteria including as S. aureus, certain Mycobacterium including M. tuberculosis, as well as certain fungi, viruses, and parasites.

### Low pH Therapeutic Benefits for Respiratory Infection and Exposure

The low pH therapeutic offers many advantages over anti-infectives for respiratory infection and exposure. These include 1) providing a single therapeutic solution effective on one or more pathogens including but not limited to bacteria, viruses, fungi, and parasites, 2) reduction in inflammation due to reduced pathogen population, reduced endotoxins, and/or reduced reaction to allergens, 3) reducing or eliminating use of antibiotics for indications reducing selection pressure on pathogens to become drug resistant and extending the useful life of traditional antibiotics, and 4) reducing the side effects of drugs such as antibiotics and corticosteroids.

One significant side effect of systemic antibiotic treatment is the dysbiosis of gastrointestinal microbiota, which has been correlated to a wide range of illness. These include gastrointestinal illnesses (such as irritable bowel syndrome, Crohn's disease, colitis, acute pancreatitis, environmental enteric dysfunction, and anorexia nervosa); mental and neurological illness (such as anxiety, depression, neuropsychiatric disorders, Parkinson disease, post-stroke neuroinflammation, hypertension, schizophrenia, autism, and spinal cord injury); coronary illness (such as right ventricular dysfunction, splanchnic congestion and heart failure); pulmonary illness (such as COPD, viral respiratory infections and neuroinflammatory disorders, allergy, asthma and obstructive sleep apnea); liver illness (nonalcoholic fatty liver disease, nonalcoholic steatohepatitis, and alcoholic hepatitis); chronic kidney disease; spondyloarthritis; and sepsis.

### Examples 39-42

### Low pH Therapeutic Efficacy on Sinusitis Bacteria

Purpose: Previous testing in demonstrated high efficacy on pathogenic Gram-negative Haemophilus influenzae (examples 39 and 40). Additional in vitro testing was performed to determine the efficacy of a low pH therapeutic formulation on Gram-negative Fusobacterium nucleatum, and Gram-positive Corynebacterium staitum, two common sinus bacteria.

Results: The results are summarized in Table 4.

TABLE 4
Low pH Efficacy on Selected Sinusitis Bacteria

| | | | pH As | pH As | Efficacy | |
|---|---|---|---|---|---|---|
| | Bacteria | Acid | Received | Applied | Log | Efficacy % |
| 39 | Haemophilus | Sulfuric | 1.871 | 1.987 | >6.94 | >99.9999% |
| | infiuenzae* | | | | | |
| 40 | Haemophilus | Sulfuric | 2.163 | 2.279 | >6.94 | >99.9999% |
| | influenzae* | | | | | |
| 41 | Fusobacterium | Sulfuric | 1.63 | 1.64 | 2.23 | 99.41% |
| | nucleatum | | | | | |
| 42 | Corynebacterium | Sulfuric | 1.63 | 1.64 | 2.96 | 99.89% |
| | staitum | | | | | |

Test conditions: Tested In Accordance With ASTM E2315, 1 minute, no soiling, non-GLP, single-replicant Bacteria tested: Haemophilus influenza ATCC 8149, Fusobacterum nucleatum ATCC 43751, Corynebacterium staitum ATCC 25586
*See US Patent Number 11,642,372 example 45 and 46

Conclusions: The low pH therapeutic formulation of 1.64 pH sulfuric acid demonstrated efficacy on Haemophilus influenzae, Fusobacterium nucleatum, and Corynebacterium staitum.

### Comparative Example: 2-4% Boric Acid Irrigate

Boric acid may be used in a concentration of 2.0-4.0% (w/v) with saline to treat chronic rhinosinusitis (Flippin, L. (2017). Methods of Treating Chronic Rhinosinusitis. U.S. Pat. No. 9,744,192), Boric acid is an extremely weak acid. At a 4.0% w/v concentration it has a pH of 4.8-5.0. The MOA for boric acid is as a toxic material to insects and microorganisms with much lower toxicity to mammals. It is generally not toxic when applied to intact skin, but it can be toxic to abraded skin,

The higher pH and different MOA in this comparative example, it does not conflict with materials as disclosed herein.

### Examples 43-46

### Low pH Therapeutic Efficacy on Sinusitis Viruses

Many acute sinus infections may begin with viral causes, and only a proportion develop a secondary bacterial infection. Rhinoviruses, influenza viruses and parainfluenza viruses are the most common causation of viral sinus infections (Brook, I. (2011). Microbiology of sinusitis, Proc Am Thorac Soc.). Table 5 provides the in vitro efficacy of a low pH therapeutic on influenza A and rhinoviruses.

TABLE 5
Efficacy of Sulfuric Acid vs Selected Sinusitis Viruses

| | | pH As | pH As | Efficacy | Efficacy |
|---|---|---|---|---|---|
| | Virus | Received | Applied | Log | % |
| 43 | Influenza A | 1.411 | 1.657 | >5 log | >99.999% |
| | virus* | | | | |
| 44 | Influenza A | 1.607 | 1.897 | >5 log | >99.999% |
| | virus* | | | | |
| 45 | Rhinovirus* | 1.258 | 1.469 | >4 log | >99.99% |
| 46 | Rhinovirus* | 1.458 | 1.6 | >4 log | >99.99% |

Test conditions:
Tested In Accordance With ASTM E1052, 1 minute, no soiling, non-GLP, single-replicant Virus tested: Influenza A (H1N1) A/PR/8/34 Strain; Rhinovirus 37
*See U.S Pat. No. 11,642,372 example 66-69

Conclusions: The low pH therapeutic demonstrated efficacy on both common bacterial and viral sinus pathogens that cause sinusitis enabling a common treatment for both bacterial and viral caused sinus infections and inflammation. This reduces the need for a clinically challenging diagnosis between bacterial and viral sinusitis causations. This may also reduce the pressure to treat viral sinusitis with antibiotics, which contributes the proliferation of AMR bacteria.

### Examples 47-48

### Low pH Therapeutic Efficacy in Sinusitis Fungi

Purpose Aspergillus fumigatus is the most common fungus associated with sinusitis and can cause disease in normal as well as the immunocompromised hosts.

Results: Table 6 provides the in vitro efficacy of a low pH therapeutic on A. fumigatus.

TABLE 6
Efficacy of Sulfuric Acid vs Sinusitis Fungi

| | | pH As | pH As | Efficacy | Efficacy |
|---|---|---|---|---|---|
| | Fungi | Received | Applied | Log | % |
| 47 | A. fumigatus* | 1.871 | 1.987 | 1.66 | 97.83% |
| 48 | A. fumigatus * | 2.163 | 2.279 | 1.7 | 98.00% |

Test conditions:
Tested In Accordance With ASTM E2315, 1 minute, no soiling, non-GLP, single-replicant Fungi tested: Aspergillus fumigatus ATCC 36607
*See U.S. Pat. No. 11,642,372 example 49 and 50

Conclusions: The low pH therapeutic demonstrated efficacy on both common bacterial, viral and fungal sinus pathogens that cause sinusitis. This enables a common treatment for both bacterial, viral and fungal caused sinus infections and inflammation. As result this reduces the need for a clinically challenging diagnosis between bacterial, viral and fungal causations and therefore may enable improved treatments to occur more quickly after the onset of symptoms.

### Example 49

In Vivo Pseudomonas aeruginosa Efficacy Study

Pseudomonas aeruginosa is a predominant organism within the hospital environment, an increasingly multidrug-resistant microbe, and the most common Gram-negative pathogen causing nosocomial pneumonia in the United States. Nosocomial pneumonia has a mortality rate ranging from 13% to 50%, lengthens hospital stays, and adds approximately US $40,000 in excess cost per patient. Nearly all P. aeruginosa infections are associated with compromised host defenses. In the lung, P. aeruginosa is known to opportunistically colonize patients with cystic fibrosis and chronic obstructive pulmonary disease. (Curran, C. et al (2018) Mechanisms and Targeted Therapies for Pseudomonas aeruginosa Lung Infection, American Journal of Respiratory and Critical Care Medicine).

Eradication of P. aeruginosa has become increasingly difficult due to its remarkable capacity to resist antibiotics. Conventional antibiotic therapies against P. aeruginosa infections have become increasingly ineffective due to the rise of multidrug-resistant strains. The overuse and misuse of antibiotics is a growing concern for public health, which can result in unnecessary side effects and the development of drug-resistant bacterial strains. Moreover, the development of new antibiotics is very limited and time consuming. Thus, the development of novel therapeutic approaches to treat P. aeruginosa infections is highly desirable and has gained more attention in the past decade. (Pang, Z. et al (2019). Antibiotic resistance in Pseudomonas aeruginosa: mechanisms and alternative therapeutic strategies, Biotechnology Advances).

After years of decreasing cases, including a significant decline in overall drug-resistant cases in 2019 compared to 2017, Multidrug Resistant (MDR) P. aeruginosa cases rose significantly in hospitals in 2020. The rate of case of hospital-onset increased 32% to 11,100 compared to 2019. People who are in the hospital or with weakened immune systems are at increased risk for P. aeruginosa infections. It is particularly dangerous for patients with chronic lung diseases. In 2020, hospitals saw higher numbers of sicker patients (hospitalization could not be avoided) who needed extended stays. This increased their risk for resistant infections. (CDC 2022 Special Report: COVID-19 U.S. Impact on Antimicrobial Resistance)

The in vitro suspension tests (examples 29 and 30) using a dilute sulfuric acid with 1.66 pH demonstrated >6 log efficacy in 1-minute on P. aeruginosa. Furthermore, the efficacy was equally high on both Antimicrobial Resistant (AMR) and non-AMR P. aeruginosa strains indicating that existing AMR protection mechanisms do not offer resistance from the low pH therapeutic, and that treatment with a low pH inhaled therapeutic has potential against P. aeruginosa in vivo including AMR strains.

Purpose: A pre-clinical in vivo study was performed to confirm that in vitro suspension efficacy of P. aeruginosa results correlates with effective in vivo treatment both for acute clinical infections and as a prophylactic to kill the pathogen in the respiratory system after exposure, but before the bacteria colony grows to become clinically significant. A murine In Vitro Infrared Spectroscopy (IVIS) protocol was selected for this study due to its ability to non-invasively quantitatively measure the infection using bioluminescent stain of P. aeruginosa.

Three groups of 5 mice were used in the study with each group was placed in separate enclosures. A drop of water containing bioluminescent P. aeruginosa was placed on each mouse's nose at hour zero. Each group was administered a nebulized solution for 10 minutes at 4, 12, 24, and 36 hours after inoculation. The first group, the negative control was administered sterile water for inhalation. The second group, the test group was administered a dilution solution of sulfuric acid (˜1.7 pH sulfuric acid and 15 ppm CaSO₄) similar to the therapeutic material used successfully in a Phase I clinical trial as outlined in U.S. Pat. No. 11,642,372. The third group, the positive control was also administered sterile water, but included administration of Ciprofloxacin, an antibiotic normally used for treating P. aeruginosa.

Results: The test group and the positive control group demonstrated effective treatment of the mice from P. aeruginosa with no deaths during the study, while several mice died in the negative control group. The study also proved that the low pH therapeutic prophylactically protected the mice from establishing an infection.

Conclusions: This study demonstrates that the low pH is effective as a both a therapeutic treatment and as a prophylactic for P. aeruginosa. Since in vitro studies have shown that the low pH therapeutic is equally effective on both AMR and non-AMR strains of P. aeruginosa, it is expected the low pH therapeutic will be effective on all strains of P. aeruginosa in vivo. Additionally, this therapeutic approach may be equally effective on a wide range of Gram-negative bacteria, Gram-positive bacteria, viruses, fungi, and parasites.

### Example 50

### In Vivo Studies of Chronic Rhinosinusitis (CRS)

Purpose: A Phase 1 clinical trial is performed that demonstrates safety of the low pH therapeutic when administered via a nebulizer for 10 minutes per treatment with 4 treatments per day for 7 days. A Phase 2 double blind clinical trial is then performed to determine the safety and efficacy on CRS patients. The efficacy endpoint was based on sinus CT imaging.

150 CRS patients are divided into 100 patients that receive the low pH therapeutic via nebulization for 10 minutes, 3 times daily for 7 days, and 50 patients that received a sterile water placebo with the same administration.

Results: The group which receives the low pH inhalation therapeutic demonstrated significant reduction in sinus inflammation and congestion as demonstrated from sinus CT imaging, while the group that receives sterile water demonstrated no statically significant improvement. No adverse events from the treatments are reported in either group.

Conclusions: This critical clinical study demonstrates that the low pH is safe and effective as treatment to reduce sinus inflammation and congestion in CRS patients. Sinusitis has multiple causes including viral, bacterial, and fungal. CRS is generally considered to be caused by bacterial infection and is often caused by AMR bacteria from previous repeated antibiotic administration.

Sinusitis from Allergies

Allergies can cause sinusitis, i.e., allergic sinusitis without any clear pathogenic cause. The low pH therapeutic that is effective in treating sinusitis resulting from a bacterial, viral, and/or fungal infection would also provide a benefit in treating allergic sinusitis. It would be expected that a reduction in pathogen population by the low pH therapeutic would result in a concomitant reduction in inflammation in a patient suffering from allergic sinusitis. Thus, a significant reduction in inflammation and symptoms can be shown by administering the low pH therapeutic to a patient suffering from allergic sinusitis.

Thus, a subject with a history of allergic sinusitis is unable to obtain relief from conventional allergy treatment, including use of a fluticasone nasal spray. The subject is treated with a low pH therapeutic of the present invention by nebulizer administration before bedtime. The following day the subject awakes with clear sinuses and is without symptoms of allergic sinusitis.

### Parasitic Nasal Pathogens

Naegleria fowleri, colloquially know as a “brain-easting amoeba” is technically not an amoeba, but a shapeshifting amoeboflagellate excavate. It is a free-living, bacteria-eating microorganism that can be pathogenic, causing an extremely rare, sudden, severe and usually fatal brain infection.

This microorganism is typically found in bodies of warm freshwater. Infections most often occur when water containing N. fowleri is inhaled through the nose, where it enters the nasal and olfactory nerve tissue, traveling to the brain through the cribriform plate

N. fowleri is sensitive to acids. The low pH therapeutic is an effective prophylactic after potential exposure to water with N. fowleri and may be an effective therapeutic after symptoms appear one to nine days after exposure.

The low therapeutic that is effective for bacterial, viral and fungal sinusitis is also effective on a rare, but deadly pathogen if given soon after exposure.

Treating Antimicrobial Resistant Respiratory Pathogens with Low pH Inhalants

The low pH therapeutic offers a novel approach to upper and lower respiratory infections. It demonstrates broad spectrum efficacy against bacteria, viruses, fungi and parasites. It demonstrates efficacy against antimicrobial resistant pathogens including WHO Priority 1: CRITICAL Pathogens for new Antibiotics.

The low pH therapeutic can be co-formulated, co-administered or used as an adjunct therapy with established respiratory and sinusitis therapeutics. These established therapeutics include antibiotics, NSAIDs, nasal decongestants, antihistamines, bronchial dilators, corticosteroids, non-steroidal anti-inflammatory sprays, zinc formulations, and Antimicrobial Peptide.

### Example 51

### Comparison of Anti-Infectives and Low pH Therapeutics

Anti-infectives such as antibiotics are widely used to treat infections. Table 7 compares anti-infectives such as antibiotics and low pH anti-bacteria, anti-viral, and anti-fungal therapeutics. Anti-infectives are effective on a limited set of pathogens using specific Mechanism of Action (MOA) targets. They are normally delivered systemically in the bloodstream though oral or intravenous administration. They generally seek to have high stability in the body, so they have sufficient time to inhibit growth of the target pathogens. The in vitro biostatic efficacy of anti-infectives is often measured using Minimum Inhibition Concentration testing over a 24-hour period. Due to their systemic distribution throughout the body, anti-infectives may have side-effects including damaging the intestinal microbiota and impacting the patient's immune system.

Low pH therapeutics have many advantages over anti-infectives by providing broad-spectrum anti-viral, anti-bacterial and anti-fungal efficacy with multiple targets. They are applied directly to infected or contaminated tissues and decompose rapidly on contact with organic material. They only have a local effect on tissues without systemic side-effects. Based on pharmacokinetics studies (Dahl, A. et al. (1983). Clearance of Sulfuric Acid-Introduced 35S from the Respiratory Tracts of Rats, Guinea Pigs and Dogs Following Inhalation or Instillation, Fundamental and Applied Toxicology) the acid anion is absorbed in the deep lung in 2-3 minutes. Neutralization occurs faster than absorption and it is expected that low pH therapeutics remains active as an antimicrobial agent for 1 minute or less. Since the hydronium API material is active in vivo for such a short period of time, the material needs to demonstrate sufficient biocidal efficacy. An appropriate in vitro efficacy test is a 1-minute Suspension Time-Kill study.

TABLE 7
Comparison of Anti-infectives and Low pH Therapeutics

| | | Low pH |
|---|---|---|
| Criteria | Anti-infectives/Antibiotics | Therapeutic |
| Spectrum of | Narrowly effective on only | Broadly effective on |
| Activity | on certain pathogens (such | wide range of |
| | as a limited set of bacteria) | pathogens including |
| | | bacteria, viruses & |
| | | fungi |
| Target Use | Internal in the bloodstream. | Applied directly to |
| | | contaminated tissues. |
| Mechanism of | Single or limited set of | Multiple targets |
| | targets | |
| Action Stability | Stable to permit uptake and | Rapidly decomposes |
| | effectiveness at infection site | on contact with |
| | | organic material |
| Potential for | High | Limited to none |
| resistance | | |
| development | | |
| Side-effects on | Several, and can negative | No systemic effects |
| humans | impact intestinal biome | |
| | and immune system | |
| In vitro | Biostatic Efficacy via | Biocidal Efficacy via |
| Efficacy Study | Minimum Inhibition | Suspension Time-Kill |
| | Concentration over 24 hours | with 1-minute contact |
| | | time |

### Cytotoxicity of Low pH Acid Formulations

Several criteria are necessary for a safe and effective low pH anti-bacterial, anti-viral and/or anti-fungal therapeutic. Many types of acids can provide hydronium concentrations sufficient to provide an anti-bacterial, anti-viral and/or anti-fungal effect. Additionally, the low pH acid formulation needs to provide no or minimum deleterious effect to host tissues. Pre-clinical and clinical studies have demonstrated that aerosolized sulfuric acid in the pH range of 1.5 to 2.0 is not harmful to host respiratory tissues in vivo. The reason for this difference in toxicity of this dilute acid between host tissues and pathogens is not known but may be the result of host tissues being less sensitive to sudden temporary increases in extra-cellular proton concentration, while the acid rapidly neutralizes in vivo.

Previous studies have demonstrated no cytotoxicity of sulfuric acid in the range of 1.7-1.5 pH to mouse fibroblast cells. However, after neutralization the residual anions from certain acids can be cytotoxic when absorbed, particularly to more sensitive human liver cells. This limits the use of many potential acids in the low pH antimicrobial respiratory inhalant.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A pharmaceutically acceptable therapeutic inhalation fluid for administration into a respiratory system of a subject comprising:
a carrier fluid; and
a pharmaceutically acceptable acid composition present in the carrier fluid in an amount sufficient to provide a solution pH between 1.5 and 2.5, the pharmaceutically acceptable acid composition exhibiting antimicrobial activity against at least one microbial pathogen when introduced into the respiratory system of a subject and further comprising at least one antimicrobial peptide.

2. The pharmaceutically acceptable therapeutic inhalation fluid of claim 1 wherein the at least one microbial pathogen is an antimicrobial-resistant pathogen.

3. The pharmaceutically acceptable therapeutic inhalation fluid of claim 2 wherein the pharmaceutically acceptable acid composition comprises a compound selected from the group consisting of sulfuric acid, hydrochloric acid and mixtures thereof.

4. The pharmaceutically acceptable therapeutic inhalation fluid of claim 2 wherein the pharmaceutically acceptable acid composition is present in the carrier fluid in an amount sufficient to provide a solution pH between 1.5 and 2.2.

5. The pharmaceutically acceptable therapeutic inhalation fluid of claim 2 wherein the pharmaceutically acceptable acid composition is present in the carrier fluid in an amount sufficient to provide a solution pH between 1.5 and 2.0.

6. The pharmaceutically acceptable therapeutic inhalation fluid of claim 5 wherein the antimicrobial peptide is selected from the group consisting of cathelicidin peptides, defensins, lactoferrin, and mixtures thereof.

7. The pharmaceutically acceptable therapeutic inhalation fluid of claim 1 consisting essentially of:
a carrier fluid; and
a pharmaceutically acceptable acid composition selected from the group consisting of sulfuric acid, hydrochloric acid and mixtures thereof, the pharmaceutically acceptable acid composition present in the carrier fluid in an amount sufficient to provide a solution pH between 1.5 and 2.5, the pharmaceutically acceptable acid composition exhibiting antimicrobial activity against at least one antimicrobial resistant pathogen when introduced into the respiratory system of the subject and at least one antimicrobial peptide.

8. The pharmaceutically acceptable therapeutic inhalation fluid of claim 7 wherein the antimicrobial peptide is selected from the group consisting of cathelicidin peptides, defensins, lactoferrin, and mixtures thereof.

9. The pharmaceutically acceptable therapeutic inhalation fluid of claim 2 further comprising at least one active pharmaceutical ingredient selected from the group consisting of adrenergic β₂ receptor agonists, steroids, non-steroidal anti-inflammatory compounds, muscarinic antagonists and mixtures thereof.

10. The pharmaceutically acceptable therapeutic inhalation fluid of claim 2 wherein the at least one microbial resistant pathogen is selected from the group consisting of Enterococcus species, Staphylococcus species, Klebsiella species, Acinetobacter species, Pseudomonas species, Enterobacter species, and mixtures thereof.

11. A method of treating a respiratory infection caused by at least one antimicrobial-resistant pathogen present in the respiratory tract of a subject, the method comprising the steps of:
introducing at least one dose of a pharmaceutically acceptable therapeutic inhalation fluid into contact with the respiratory tract of a subject presenting with a respiratory infection, the pharmaceutically acceptable therapeutic inhalation fluid comprising a fluid carrier and a pharmaceutically acceptable acid composition active against at least one antimicrobial resistant pathogen present tin the respiratory tract of the subject, the pharmaceutically acceptable acid composition present in an amount sufficient to provide a pH between 1.5 and 2.5 and at least one antimicrobial peptide.

12. The method of claim 11 wherein the at least one dose of the pharmaceutically acceptable therapeutic inhalation fluid is introduced into the subject at a temperature between 50° F. and 150° F. at a particle size between 0.1 and 0.5 microns.

13. The method of claim 11 wherein the at least one dose is introduced into the respiratory tract of the subject via the oral cavity via inhalation.

14. The method of claim 11 further comprising the step of loading the at least one dose of the pharmaceutically acceptable therapeutic inhalation fluid into a receptacle associated with one of a nebulizer, vaporizer, or humidifier.

15. The method of claim 14 wherein the at least one dose of the pharmaceutically acceptable therapeutic inhalation fluid is present in a liquid volume between 1 and 15 ml in the receptacle and is introduced into the respiratory tract of the subject via the oral cavity via inhalation at a particle size between 0.1 and 0.5 microns over an interval between 30 second and 20 minutes.

16. The method of claim 11 wherein the pharmaceutically acceptable therapeutic inhalation fluid comprising the fluid carrier and the pharmaceutically acceptable acid composition active against at least one antimicrobial resistant pathogen present in the respiratory tract of the subject is selected from the group consisting of sulfuric acid, hydrochloric acid and mixtures thereof.

17. The method of claim 11 wherein the pharmaceutically acceptable therapeutic inhalation fluid has a solution pH between 1.5 and 2.2.

18. The method of claim 11 wherein the pharmaceutically acceptable therapeutic inhalation fluid has a solution pH between 1.5 and 2.0.

19. The method of claim 18 wherein the pharmaceutically acceptable therapeutic inhalation fluid further comprises at least one active pharmaceutical ingredient selected from the group consisting of adrenergic β₂ receptor agonists, steroids, non-steroidal anti-inflammatory compounds, muscarinic antagonists, and mixtures thereof.

21. The method of claim 17 wherein the pharmaceutically acceptable therapeutic inhalation fluid consists of the carrier fluid and the pharmaceutically acceptable acid composition selected from the group consisting of hydrochloric acid, sulfuric acid and mixtures thereof.

22. The method of claim 11, further comprising the step of:
introducing at least one dose of a pharmaceutically acceptable therapeutic inhalation fluid and at least one antimicrobial peptide selected from the group consisting of cathelicidin peptides, defensins, lactoferrin, and mixtures thereof into contact with the respiratory tract of the subject presenting with a respiratory infection.

23. A system for treating a respiratory infection caused by at least one antimicrobial-resistant pathogen, the system comprising:
a medication delivery device, the medication delivery device including at least one a medication storage chamber and a medication outlet member in fluid communication with the medication storage chamber; and
a pharmaceutically acceptable therapeutic inhalation fluid composition contained in the medication storage chamber and deliverable through the medication outlet member, the pharmaceutically acceptable therapeutic inhalation fluid composition comprising:
a fluid carrier; and
a pharmaceutically acceptable acid composition present in the carrier fluid in an amount sufficient to provide a solution pH between 1.5 and 2.5, the pharmaceutically acceptable acid composition exhibiting antimicrobial activity against at least one microbial pathogen when introduced into the respiratory system of a subject and further comprising at least one antimicrobial peptide;
wherein at least one portion of the pharmaceutically acceptable therapeutic inhalation fluid composition is dispatched through the medication outlet member in a vaporized or atomized state.

24. The system of claim 23 wherein the medication delivery device includes at least two chambers and wherein the pharmaceutically acceptable acid composition is maintained in one first medication chamber and the at least one antimicrobial peptide is maintained in a second medication chamber.

25. The system of claim 24 wherein the medication delivery device further comprises at least one mixing apparatus, the at least one mixing apparatus in communication with the first medication chamber and the second medication chamber, the mixing apparatus communicating with the medication outlet member
